(19) United States
(12) Patent Application Publication
(10) Pub. No.: US 2026/0251550 A1
(43) Pub. Date: Aug. 27, 2026

(54) METHODS AND RELATED ASPECTS FOR CHARACTERIZING LABELED NANOPARTICLES

(71) Applicant: THE JOHNS HOPKINS UNIVERSITY, Baltimore, MD (US)

(72) Inventors: Tza-Huei Jeff WANG, Germantown, MD (US); Hai-Quan MAO, Baltimore, MD (US); Sixuan LI, Baltimore, MD (US); Yizong HU, Baltimore, MD (US); Andrew LI, Baltimore, MD (US)

(21) Appl. No.: 18/877,461

(22) PCT Filed: Jun. 22, 2023
(86) PCT No.: PCT/US2023/025916

§ 371 Date: Dec. 20, 2024

Related U.S. Application Data
(60) Provisional application No. 63/354,672, filed on Jun. 22, 2022.

(51) Int. Cl. G01N 15/14; A61K 48/00; G01N 15/00

(52) U.S. Cl. CPC G01N 15/1459; A61K 48/0041; G01N 2015/0038; G01N 2015/0053; G01N 2015/1493

(57) ABSTRACT

Provided herein are methods of characterizing a nanoparticle in a population of labeled nanoparticles. In some embodiments, the methods include determining size measures of the labeled nanoparticles from retention data and payload properties of the labeled nanoparticles from the signal data. Related kits, systems, and computer readable media are also provided.

### CROSS-REFERENCE TO RELATED APPLICATIONS

This application is the national stage entry of International Patent Application No. PCT/US2023/025916, filed on Jun. 22, 2023, and published as WO 2023/250053 A1 on Dec. 28, 2023, which claims the benefit of U.S. Provisional Patent Application Ser. No. 63/354,672, filed on Jun. 22, 2022, which are hereby incorporated by reference in their entireties.

### STATEMENT REGARDING FEDERALLY SPONSORED RESEARCH OR DEVELOPMENT

This invention was made with Government support under grant nos. AI137272 and AI155313, awarded by the National Institutes of Health. The Government has certain rights in the invention.

### BACKGROUND

Lipid nanoparticles (LNPs) formulated from a mixture of an ionizable lipid, a helper lipid, cholesterol, a PEG lipid, and therapeutic nucleic acids have been shown to be potent and safe prophylactic vaccines and therapeutic delivery vehicles. For example, two mRNA vaccines against COVID-19 have received full FDA approval, and positive therapeutic outcomes were reported in a phase 1 clinical trial for transthyretin amyloidosis in which CRISPR-Cas9 mRNA and a single guide RNA were co-delivered to the liver. Along with successful applications, there have been efforts in investigating the packaging characteristics of LNPs. Through experiments with siRNA-, mRNA-, and plasmid DNA (pDNA)-loaded LNPs, some features of these vehicles have been reported previously, including the assembled structures, interior location of cargos, lipid compositions, and dynamic behaviors during the purification process. They provide better understandings of the structure-property-function relationship that may direct further optimization of LNP designs.

A typical formulation process for mRNA LNPs starts with rapid mixing of an aqueous solution of mRNA and an alcohol solution of lipids at a pH, e.g., 4.0, that is substantially lower than the pKa of the ionizable lipid, which is typically around 6.5. Cryo-EM showed that different LNP species, vesicular or solid, are formed under this condition. During dialysis against a buffer at the physiological pH of 7.4, the ionizable lipids lose most of their positive charges (i.e., deprotonation) and form a hydrophobic, amorphous core, rendering an electron-dense appearance to all LNPs under cryo-EM. It has been difficult to characterize the payload distribution and capacity in these LNPs at a single-nanoparticle level, primarily due to a technical gap for distinguishing empty LNPs from those with a payload and quantifying mRNA molecules in mRNA-loaded LNPs by cryo-EM, or by other methods such as small-angle neutron scattering, NMR, and nanoparticle tracking analysis. However, payload distribution and capacity are important characteristics to assess, because they hint at molecular assembly mechanism of mRNA LNPs and influence their pharmacodynamics, pharmacokinetics, and delivery efficiency.

Accordingly, there is a need for additional methods, and related aspects, for characterizing non-viral vectors, including LNPs and other nanoparticles.

### SUMMARY

The present disclosure relates, in certain aspects, to methods for characterizing non-viral vectors, including LNPs, polymer nanoparticles, inorganic or organic nanoparticles, extracellular vesicles, and liposomes. In some aspects, for example, the present disclosure provides a multi-color fluorescence spectroscopic technique that integrates a single-molecule detection (SMD) platform, fluorescence coincidence analysis, and a quantitative fluorescence deconvolution algorithm for characterization of the payload distribution and capacity of mRNA LNPs. In some embodiments, the SMD platform, sometimes referred to herein as cylindrical illumination confocal spectroscopy (CICS), features a single-fluorophore sensitivity and near 100% mass detection efficiency owing to uniform fluorescent excitations by its one-dimensional laser beam shaping. This flow-based technique allows for the detection of an entire nanoparticle population passing through the detector. In some embodiments, by fluorescently labeling different species or components in LNP formulations, and subsequently analyzing the coincidence of the single-particle fluorescence signals, all species or components in an mRNA LNP formulation, for example, can be differentiated from one another. The methods and related aspects disclosed herein can also be used to quantify the mRNA or other payload distribution and capacity at single-particle resolution through a deconvolution algorithm of the fluorescence signal distribution of payload-carrying LNPs against that of free payload molecules. These and other aspects will be apparent upon a complete review of the present disclosure, including the accompanying figures.

In one aspect, the present disclosure provides a method of characterizing a nanoparticle in a population of labeled nanoparticles. The method includes determining a retention time taken for the nanoparticle to flow from a first point in or proximal to a fluidic channel to or through a detection zone of the fluidic channel when a fluidic sample that comprises the population of labeled nanoparticles flows through the fluidic channel to produce retention data. The method also includes determining a size measure of the nanoparticle from the retention data to produce nanoparticle size data, detecting a detectable signal produced by one or more labels of one or more components of the nanoparticle when the nanoparticle flows through the detection zone of the fluidic channel to produce signal data, and determining at least one payload property of the nanoparticle from the signal data to produce nanoparticle payload property data, thereby characterizing the nanoparticle in the population of labeled nanoparticles. In some embodiments, the population of labeled nanoparticles comprises lipid nanoparticles (LNPs).

In some embodiments, the first point in or proximal to the fluidic channel comprises an inlet to the fluidic channel. In some embodiments, at least two, at least three, or more components of the nanoparticles in the population of nanoparticles comprise different labels from one another. In some embodiments, the different labels comprise different fluorescent labels.

In some embodiments, the nanoparticle size measure comprises a diameter of the nanoparticle. In some embodiments, the method comprises characterizing a plurality of the nanoparticles in the population of labeled nanoparticles in the fluidic sample. In some embodiments, the method comprises producing the nanoparticle size data and the nanoparticle payload property data substantially simultaneously.

In some embodiments, at least a first component of the nanoparticles in the population of labeled nanoparticles comprises a payload molecule. In some embodiments, the payload molecule comprises a therapeutic agent. In some embodiments, the payload molecule comprises a metabolite, a nucleic acid, and/or a polypeptide. In some embodiments, at least a second component of the nanoparticles in the population of labeled nanoparticles comprises: a polymer, a copolymer, a lipid, a fluorophore, a carbohydrate, an emulsion, a vesicle, a cell, polyethylene glycol (PEG), cholesterol, a liposome, a carbon nanotube, silica, and gold.

In some embodiments, the method comprises flowing the fluidic sample through the fluidic channel such that the population of labeled nanoparticles flow through the detection zone of the fluidic channel. In some embodiments, the method comprises illuminating the detection zone of the fluidic channel substantially uniformly across an entire cross section of the fluidic channel such that each of the nanoparticles in the population of labeled nanoparticles passes through illumination light upon passing through the detection zone. In some embodiments, the method comprises detecting each of the nanoparticles in the population of labeled nanoparticles based on corresponding responses to the illuminating to determine retention times taken for each of the labeled nanoparticles to flow from the first point in or proximal to the fluidic channel to or through the detection zone.

In some embodiments, the at least one payload property of the nanoparticle comprises an amount of payload molecule, an encapsulation efficiency measure, and/or payload molecule capacity of the nanoparticle. In some embodiments, the retention data comprises a measure of hydrodynamic separation between the nanoparticle and at least one other nanoparticle in the population of labeled nanoparticles. In some embodiments, the signal data comprises a relative signal intensity and/or a coincidence measure of detectable signals produced by different labels of at least two components of the nanoparticles in the population of the labeled nanoparticles.

In some embodiments, the method comprises distinguishing nanoparticles comprising a payload molecule from nanoparticles lacking a payload molecule in the population of the labeled nanoparticles using at least the signal data. In some embodiments, the method comprises determining a distribution of the at least one payload property of the nanoparticles in the population of labeled nanoparticles. In some embodiments, the method further comprises determining a concentration of nanoparticles in the population of labeled nanoparticles using at least the signal data. In some embodiments, the method comprises separating the population of labeled nanoparticles into at least two selected fractions before, during, or after characterizing the nanoparticle in the population of labeled nanoparticles.

In another aspect, the present disclosure provides a method of characterizing a non-viral vector in a population of labeled non-viral vectors. The method includes determining a retention time taken for the non-viral vector to flow from a first point in or proximal to a fluidic channel to or through a detection zone of the fluidic channel when a fluidic sample that comprises the population of labeled non-viral vectors flows through the fluidic channel to produce retention data. The method also includes determining a size measure of the non-viral vector from the retention data to produce non-viral vector size data, and detecting a detectable signal produced by one or more labels of one or more components of the non-viral vector when the non-viral vector flows through the detection zone of the fluidic channel to produce signal data. In addition, the method also includes determining at least one payload property of the non-viral vector from the signal data to produce non-viral vector payload property data, thereby characterizing the non-viral vector in the population of labeled non-viral vectors. Related kits, systems, and computer readable media are also provided. Non-limiting examples of non-viral vectors that are optionally used or adapted for use with the methods and other aspects of the present disclosure, include extracellular vesicles (EV) (e.g., biological nanoparticles naturally secreted by cells, etc.), liposomes (e.g., ligand-targeting liposomes, stimulus-responding liposomes, etc.), polymers (e.g., dendrimers, polyethylenimine, chitosan, polylactic acid/poly (lactic-co-glycolic acid), amino acid derived biopolymers (e.g., polyamides(PA)s, polyesters(PE)s, poly(ester-amide)s(PEA)s, polyurethanes(PU)s, and poly (depsipeptide)s (PDP)s, among many others), alginate, etc.), cochleates, carbon nanotubes, nanoparticles (e.g., lipid nanoparticles (LNPs), mesoporous silica nanoparticles, gold nanoparticles, etc.), and combinations thereof, among others.

In another aspect, the present disclosure provides a kit that includes a device comprising a fluidic channel having a detection zone, and instructions for using the device to: determine a retention time taken for a nanoparticle to flow from a first point in or proximal to the fluidic channel to or through the detection zone of the fluidic channel when a fluidic sample that comprises a population of labeled nanoparticles flows through the fluidic channel to produce retention data, determine a size measure of the nanoparticle from the retention data to produce nanoparticle size data, detect a detectable signal produced by one or more labels of one or more components of the nanoparticle when the nanoparticle flows through the detection zone of the fluidic channel to produce signal data, and determine at least one payload property of the nanoparticle from the signal data to produce nanoparticle payload property data. In some embodiments, the kit further comprises instructions for using the device to determine a concentration of nanoparticles in the population of labeled nanoparticles using at least the signal data. In some embodiments, the device comprises a microfluidic device. In some embodiments, the fluidic channel comprises a capillary tube.

In another aspect, the present disclosure provides a system for characterizing a nanoparticle in a population of labeled nanoparticles. The system includes a device receiving area configured to receive a device comprising a fluidic channel having a detection zone, and a fluid handling apparatus configured to effect a flow of a fluidic sample that comprises the population of labeled nanoparticles through the detection zone when the fluid handling apparatus is operably connected to the device and when the fluidic sample is disposed in the fluidic channel. The system also includes a light source configured to introduce an incident light toward the detection zone when the device is received in the device receiving area, and a detector configured to detect one or more detectable signals produced in the detection zone when the fluid handling apparatus effects the flow of the population of labeled nanoparticles flow through the fluidic channel and when the device is received in the device receiving area. In addition, the system also includes a controller that comprises, or is capable of accessing, computer readable media comprising non-transitory computer-executable instructions which, when executed by at least one electronic processor, perform at least: flowing the fluidic sample through the fluidic channel using the fluid handling apparatus such that the population of labeled nanoparticles flow through the detection zone of the fluidic channel, introducing the incident light from the light source toward the detection zone when the device is received in the device receiving area, determining a retention time taken for the nanoparticle to flow from a first point in or proximal to a fluidic channel to or through the detection zone of the fluidic channel when the population of labeled nanoparticles flows through the fluidic channel to produce retention data, determining a size measure of the nanoparticle from the retention data to produce nanoparticle size data, detecting a detectable signal produced by one or more labels of one or more components of the nanoparticle when the nanoparticle flows through the detection zone of the fluidic channel to produce signal data, and determining at least one payload property of the nanoparticle from the signal data to produce nanoparticle payload property data to thereby characterize the nanoparticle in the population of labeled nanoparticles.

In another aspect, the present disclosure provides a computer readable media comprising non-transitory computer executable instruction which, when executed by at least electronic processor, perform at least: determining a retention time taken for a nanoparticle to flow from a first point in or proximal to a fluidic channel to or through a detection zone of the fluidic channel when a fluidic sample that comprises a population of labeled nanoparticles flows through the fluidic channel to produce retention data, determining a size measure of the nanoparticle from the retention data to produce nanoparticle size data, detecting a detectable signal produced by one or more labels of one or more components of the nanoparticle when the nanoparticle flows through the detection zone of the fluidic channel to produce signal data, and determining at least one payload property of the nanoparticle from the signal data to produce nanoparticle payload property data.

In some embodiments of the system or computer readable media, the population of labeled nanoparticles comprises lipid nanoparticles (LNPs). In some embodiments of the system or computer readable media, the device comprises a microfluidic device. In some embodiments of the system or computer readable media, the fluidic channel comprises a capillary tube. In some embodiments of the system or computer readable media, the light source comprises a cylindrical illumination apparatus.

In some embodiments of the system or computer readable media, at least two, at least three, or more components of the nanoparticles in the population of nanoparticles comprise different labels from one another. In some embodiments of the system or computer readable media, the different labels comprise different fluorescent labels.

In some embodiments of the system or computer readable media, the nanoparticle size measure comprises a diameter of the nanoparticle. In some embodiments of the system or computer readable media, In some embodiments of the system or computer readable media, the non-transitory computer-executable instructions which, when executed by the electronic processor, further perform at least: determining a concentration of nanoparticles in the population of labeled nanoparticles using at least the signal data. In some embodiments of the system or computer readable media, the non-transitory computer-executable instructions which, when executed by the electronic processor, further perform at least: characterizing a plurality of the nanoparticles in the population of labeled nanoparticles in the fluidic sample. In some embodiments of the system or computer readable media, the non-transitory computer-executable instructions which, when executed by the electronic processor, further perform at least: producing the nanoparticle size data and the nanoparticle payload property data substantially simultaneously.

In some embodiments of the system or computer readable media, at least a first component of the nanoparticles in the population of labeled nanoparticles comprises a payload molecule. In some embodiments of the system or computer readable media, the payload molecule comprises a therapeutic agent. In some embodiments of the system or computer readable media, the payload molecule comprises a metabolite, a nucleic acid, and/or a polypeptide. In some embodiments of the system or computer readable media, at least a second component of the nanoparticles in the population of labeled nanoparticles comprises a molecule selected from the group consisting of: a polymer, a copolymer, a lipid, a fluorophore, a carbohydrate, an emulsion, a vesicle, a cell, polyethylene glycol (PEG), cholesterol, a liposome, a carbon nanotube, silica, and gold.

In some embodiments of the system or computer readable media, the non-transitory computer-executable instructions which, when executed by the electronic processor, further perform at least: illuminating the detection zone of the fluidic channel substantially uniformly across an entire cross section of the fluidic channel such that each of the nanoparticles in the population of labeled nanoparticles passes through illumination light upon passing through the detection zone. In some embodiments of the system or computer readable media, the non-transitory computer-executable instructions which, when executed by the electronic processor, further perform at least: detecting each of the nanoparticles in the population of labeled nanoparticles based on corresponding responses to the illuminating to determine retention times taken for each of the labeled nanoparticles to flow from the first point in or proximal to the fluidic channel to or through the detection zone. In some embodiments of the system or computer readable media, the at least one payload property of the nanoparticle comprises an amount of payload molecule, an encapsulation efficiency measure, and/or payload molecule capacity of the nanoparticle. In some embodiments of the system or computer readable media, the retention data comprises a measure of hydrodynamic separation between the nanoparticle and at least one other nanoparticle in the population of labeled nanoparticles. In some embodiments of the system or computer readable media, the signal data comprises a relative signal intensity and/or a coincidence measure of detectable signals produced by different labels of at least two components of the nanoparticles in the population of the labeled nanoparticles.

In some embodiments of the system or computer readable media, the non-transitory computer-executable instructions which, when executed by the electronic processor, further perform at least: distinguishing nanoparticles comprising a payload molecule from nanoparticles lacking a payload molecule in the population of the labeled nanoparticles using at least the signal data. In some embodiments of the system or computer readable media, the non-transitory computer-executable instructions which, when executed by the electronic processor, further perform at least: determining a distribution of the at least one payload property of the nanoparticles in the population of labeled nanoparticles. In some embodiments of the system or computer readable media, the non-transitory computer-executable instructions which, when executed by the electronic processor, further perform at least: separating the population of labeled nanoparticles into at least two selected fractions before, during, or after characterizing the nanoparticle in the population of labeled nanoparticles. In some embodiments of the system or computer readable media, the non-transitory computer-executable instructions which, when executed by the electronic processor, further perform at least: determining an encapsulation efficiency measure of nanoparticles in the population of labeled nanoparticles.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of characterizing a nanoparticle in a population of labeled nanoparticles according to some aspects disclosed herein.

FIG. 2 is a schematic diagram of an exemplary system suitable for use with certain aspects disclosed herein.

FIGS. 3A-3H. Instrumentation of multi-color CICS platform, methodology for characterization of LNP formulations, and fluorescent compensation. (a) Species of interest in LNP formulations include the mRNA-loaded LNPs, empty LNPs, and free mRNAs. Three fluorescent tags were used for the single-particle fluorescence detection and species classification: all mRNAs have Cy5 tags; 5% of the helper lipids carry a TMR tag; YOYO-1 was added into the LNP sample prior to CICS assessment to stain free mRNAs. (b) Instrumental setup of three-color CICS. The lasers were first combined by a beam combiner to give a single output which allows the fluorescence coincidence detection. The laser beam was further expanded in one dimension by a cylindrical lens (CL), which gave an observation volume that covers the whole cross-section of the capillary channel. Such design allows CICS to obtain nearly 100% mass detection efficiency. The rectangular confocal aperture (CA) rejects the out-of-plane signal and confine the signal collection only from the center of the illumination volume, which renders highly uniform fluorescent signals. Each particle that passed through the detection volume generated a unique fluorescence signal that was recorded by single-photon counting avalanche photodiodes (APDs). (c) The single-particle fluorescence trace was processed with a thresholding algorithm to identify all the burst events. Based on the fluorescent coincidence across the three colors, the fluorescence was classified as: mRNA-loaded LNPs (circles, TMR-Cy5 coincident), empty LNPs (crosses, TMR only), and free mRNAs (asterisks, Cy5-YOYO-1 coincident). (d) The statistical distribution of Cy5-to-TMR intensity ratio of the spill-over signals, justifying a fix-ratio compensation with a factor of 0.116. (e) Application of compensation successfully corrected most of the spill-over signals from TMR channel to Cy5 channel. (f) The Cy5 intensity profile of single free mRNA molecules, and theoretical Cy5 intensity profiles of multiplexed mRNAs expected in LNPs, compared with the histogram obtained from an LNP sample containing a distribution of the mRNA payload shown in (g). (h) TMR intensity profiles of LNP formulations correlate with their relative helper lipid content.

FIGS. 4A-4I. mRNA payload behaviors of a benchmark mRNA LNP formulation (DLin-MC3-DMA:DSPC:cholesterol:DMG-PEG=50:10:38.5:1.5) (a) Example of 3-color raw signals at pH 4.0. Circles label events of lipophilic complexes; Asterisks label events of non-lipophilic complexes; Crosses label events of empty LNPs. (b) Example of 3-color raw signals upon dialysis to pH 7.4. Asterisks label events of free mRNAs in panel 1; Circles label events of mRNA-loaded LNPs in panel 2; Crosses label events of empty LNPs in panel 3. In (a) and (b), the dashed lines show the threshold set for detection. (c) Classification of LNP species into empty LNPs (upper-left quadrant), lipophilic complexes (upper-right quadrant) and non-lipophilic complexes (lower-right quadrant) by plotting TMR signal intensity against Cy5 signal intensity at pH 4.0. 10% of 141,530 signals are shown in the figure. (d) Classification of LNP species into empty LNPs (upper-left quadrant), mRNA-loaded LNPs (upper-right quadrant), and free mRNAs (lower-right quadrant) detected at pH 7.4. For clarity, 10% of 195,090 signals are shown in the figure. The percentages labeled are relative to all TMR events. Free mRNA events accounted for only 4% of all events. (e) Identification of mRNAs that were encapsulated in LNPs thus inaccessible to YOYO-1 and un-encapsulated ones at pH 7.4 by plotting YOYO-1 signal intensity to Cy5 signal intensity. For clarity, 10% of 71,320 signals are shown in this figure. The upper-left quadrant population was presumably empty LNPs non-specifically tagged by YOYO-1. The percentages labeled are relative to all Cy5 events. (f) Application of 3-color authentication for population classification reduced frequency of false mRNA-loaded LNP signals from 2-color authentications. (g) TMR signal intensity profiles of LNP species at pH 4.0 or 7.4. (h) Cy5 signal intensity profiles of LNP species at pH 4.0 or 7.4. (i) Calculated mRNA payload distributions of this benchmark mRNA LNP formulation using deconvolution algorithm.

FIGS. 5A-5J. Effects of molar ratio of DMG-PEG on the payload capacity and lipid content of mRNA LNPs (DLin-MC3-DMA:DSPC:cholesterol:DMG-PEG=50:10:40-x:x). (a) The z-average particle diameter of mRNA LNPs assessed by dynamic light scattering (DLS). (b-d) The mRNA payload distribution profiles of formulations at (b) pH 7.4; (c) pH 4.0 for lipophilic complexes; or (d) pH 4.0 for non-lipophilic complexes. (e) The number average mRNA copy per LNP. (f, g) The geometric mean of TMR signals (indicator of relative helper lipid content) of (f) lipophilic complexes at pH 4.0 and mRNA-loaded LNPs at pH 7.4; or (g) empty LNPs at either pH 4.0 or 7.4. (h) The fraction of empty LNPs. (i) The absolute number concentrations of mRNA-loaded or empty LNPs at pH 7.4. (j) The average fold change of mRNA payload and helper lipid content from lipophilic complexes at pH 4.0 to mRNA-loaded LNPs at pH 7.4. The consistently higher fold change of helper lipid content indicated that merge of empty LNPs to lipophilic complexes occurred. All error bars in this figure represent standard deviations of 3 independent experiments (formulating LNPs from raw materials and then applying CICS analysis).

FIGS. 6A and 6B. Mechanisms of determination of payload capacity and distribution of mRNA LNPs by the PEG content. (a, b) The hypothesized assembly processes and characteristics of LNP formulation with a high concentration of PEG mol % (a); or a low concentration of PEG mol % (b) and composition drift during dialysis from pH 4.0 (left) to pH 7.4 (right). The populational frequencies labeled are real data from the formulation with PEG mol %=1.5% (a) or 0.5% (b). In (a), each number label represents a populational behavior during dialysis: 1, splitting of empty LNPs; 2, stabilization of empty LNPs; 3, splitting of lipophilic complexes with an initially high mRNA payload; 4, remaining a same mRNA payload for lipophilic complexes with an initially low or intermediate payload; 5, merge of empty LNPs with mRNA complexes; 6, merge of non-lipophilic complexes. In (b), the labels are: 1, merge between lipophilic complexes; 2, merge of empty LNPs with mRNA complexes; 3, merge of non-lipophilic complexes; 4, splitting of empty LNPs.

FIGS. 7A-7J. Effects of N/P ratio on the payload capacity and lipid content of mRNA LNPs. (a) The z-average particle size of mRNA LNPs assessed by dynamic light scattering (DLS). (b-d) The mRNA payload distribution profiles of formulations at pH 7.4 (b); pH 4.0 for lipophilic complexes (c); or pH 4.0 for non-lipophilic complexes (d). (e) The number average mRNA copy per LNP at either pH 4.0 or pH 7.4. (f, g) Geometric mean of TMR signals (indicator of relative helper lipid content) of lipophilic complexes at pH 4.0 and mRNA-loaded LNPs at pH 7.4 (f), or empty LNPs at either pH 4.0 or 7.4 (g). (h) The fraction of empty LNPs assessed at either pH 4.0 or 7.4. All error bars in this figure represent standard deviations from 3 independent experiments (formulating LNPs from raw materials and then applying CICS analysis).

FIGS. 8A and 8B. Mechanisms of determination of payload capacity and distribution of mRNA LNPs by N/P ratio. (a, b) The hypothesized assembly processes and characteristics of LNP formulation with a high N/P ratio (a); or a low N/P ratio (b) and composition drift during dialysis from pH 4.0 (left) to pH 7.4 (right). At pH 4.0, the populational frequencies labeled are real data for an N/P ratio of 12 (a) or 2 (b). Labels in both (a) and (b): 1, a kinetically favorable (major) process; 2, a kinetically unfavorable (minor) process. At pH 7.4, the mRNA-loaded LNPs presumably hold the same size because the relative ratio of PEG lipid to all lipids is the same.

FIGS. 9A-9M. Effects of lipid and mRNA concentrations and mRNA size on payload capacity and distribution, and effect of empty LNPs on mRNA delivery efficiency. (a-c) The effect of mRNA (and lipids) concentration on (a) the z-average particle size and number-average mRNA payload; (b) the payload distribution; and (c) the relative helper lipid content in mRNA-loaded or empty LNPs, as well as the fraction of empty LNPs at pH 7.4. (d-j) Effect of mRNA size (996 nt vs. 1929 nt) on (d) the z-average particle size, (e) the number-average mRNA payload, (f) the payload distribution at pH 4.0 for lipophilic complexes; (g) the payload distribution at pH 4.0 for non-lipophilic complexes; (h) the payload distribution at pH 7.4; (i) the relative helper lipid content of mRNA-loaded LNPs at pH 7.4; and (j) the fraction of empty LNPs at pH 7.4. (k-m) Effect of empty LNP content on mRNA delivery efficiency. The IVIS images of mice and harvested livers and spleens at 12 h post-i.v. injection of LNP formulations at an mRNA dose of 0.5 mg mRNA/kg. The harvested organs were subsequently homogenized with the local luciferase concentration measured by ex vivo bioluminescence assay, and the results are shown in (l) for the liver and (m) for the spleen. For (k-m), N/P=3 or 6 means the LNPs were directly formulated with an N/P ratio of 3 or 6, while N/P=3+3 means the LNPs were first formulated with an N/P ratio of 3, and then empty LNPs containing a lipid mass that equals to the mass correlating with an N/P ratio of 3 were added and mixed with the base. This N/P=3+3 group contained ~2-fold of empty LNPs than the N/P=3 group, while keeping the population of mRNA-loaded LNPs consistent. For statistically analysis in (l) and (m), an unpaired t test was performed with * denoting p (two sided)<0.05 and *** denoting p (two sided)<0.001.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, kits, computer readable media, devices, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Biomolecule: As used herein, “biomolecule” refers to an organic molecule produced by a living organism. Examples of biomolecules, include macromolecules, such as nucleic acids, proteins, carbohydrates, and lipids.

Detect: As used herein, “detect,” “detecting,” or “detection” refers to an act of determining the existence or presence of one or more target biomolecules (e.g., nucleic acids, proteins, etc.) in a sample.

Detectable Signal: As used herein, “detectable signal” refers to signal output at an intensity or power sufficient to be detected in a given detection system. In certain embodiments, a detectable signal is emitted from a label (e.g., a fluorescent label or the like) associated with a given component of a nanoparticle.

Label: As used herein, “label” refers to a moiety attached (covalently or non-covalently), or capable of being attached, to a molecule, which moiety provides or is capable of providing information about the molecule (e.g., descriptive, identifying, etc. information about the molecule). Exemplary labels include donor moieties, acceptor moieties, fluorescent labels, non-fluorescent labels, calorimetric labels, chemiluminescent labels, bioluminescent labels, radioactive labels, mass-modifying groups, antibodies, antigens, biotin, haptens, and enzymes (including, e.g., peroxidase, phosphatase, etc.).

Nanoparticle: As used herein, “nanoparticle” in the context of non-viral vectors refers a vector or carrier that is used to deliver payload molecules to target cells or tissues in vivo, ex vivo, or in vitro. Nanoparticles are typically composed of one or more component molecules or compounds, including various lipids (e.g., ionizable lipids, helper lipids, etc.), other organic molecules (e.g., biomolecules, etc.), and/or labels (e.g., fluorescent labels, etc.), among other components.

Non-Viral Vector As used herein, “non-viral vector” in the context of payload molecule delivery vectors or carriers refers to a vector or carrier that does not involve the use of a virus. Examples of non-viral vector include extracellular vesicles, liposomes, polymers, cochleates, carbon nanotubes, nanoparticles (e.g., lipid nanoparticles (LNPs), mesoporous silica nanoparticles, gold nanoparticles, etc.), and combinations thereof, among others.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs or modified nucleotides (e.g., bromodeoxyuridine (BrdU), 2′-O-methyl modified nucleotides, 2′-fluoro modified nucleotides, etc.), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, miRNA, siRNA, shRNA, mRNA, or any combination thereof.

Payload Molecule: As used herein, “payload molecule” in the context of non-viral vectors refers a molecule, such as a biomolecule (e.g., a protein or a nucleic acid), small molecule, or other compound that can be delivered to a target cell or tissue using a given non-viral vector. Payload molecules are used in various applications, including therapeutic and analytical processes.

Protein: As used herein, “protein” or “polypeptide” refers to a polymer of at least two amino acids attached to one another by a peptide bond. Examples of proteins include enzymes, hormones, antibodies, and fragments thereof.

Sample: As used herein, “sample” means anything capable of being analyzed by the methods, cartridges and/or devices disclosed herein. Samples can include a tissue or organ from a subject; a cell (either within a subject, taken directly from a subject, or a cell maintained in culture or from a cultured cell line); a ceil lysate (or lysate fraction) or cell extract; or a solution containing one or more biomolecules derived from a cell or cellular material (e.g., a nucleic acid, a protein, etc.), which is assayed as described herein. A sample may also be any body fluid or excretion (for example, but not limited to, blood, urine, stool, saliva, tears, bile) that contains cells, cell components, or non-cellular fractions. Additional examples of samples include environment and forensic samples. Samples can also include infectious disease agents (e.g., bacteria, viruses, etc.) or plant matter, among other sample types.

System: As used herein, “system” in the context of analytical instrumentation refers a group of objects and/or devices that form a network for performing a desired objective.

Value: As used herein, “value” or “measure” generally refers to an entry in a dataset that can be anything that characterizes the feature to which the value refers. This includes, without limitation, numbers, words or phrases, symbols (e.g., + or −) or degrees.

### DETAILED DESCRIPTION

Non-viral vector, such as lipid nanoparticles (LNPs) are effective vehicles to deliver payload molecules, including mRNA vaccines and therapeutics. It has been challenging to assess mRNA packaging characteristics in LNPs, including payload distribution and capacity, which are important to understanding structure-property-function relationships for further carrier development. Accordingly, in some aspects, the present disclosure provides methods based on a multi-laser cylindrical illumination confocal spectroscopy (CICS) technique to examine mRNA and lipid contents in LNP formulations at the single-nanoparticle level. By differentiating unencapsulated mRNAs, empty LNPs and mRNA-loaded LNPs via coincidence analysis of fluorescent tags on different LNP components, and quantitatively resolving single-mRNA fluorescence, the present inventors discovered that a benchmark formulation contains mostly two mRNAs per loaded LNP, with 80% of all LNPs being empty. Systematic analysis of different formulations with control variables revealed a kinetically controlled assembly mechanism that governs the payload distribution and capacity in LNPs. These results form the foundation for a holistic understanding of the molecular assembly of mRNA LNPs. These and other aspects of the present disclosure will be apparent upon a complete review of the specification, including the accompanying drawings.

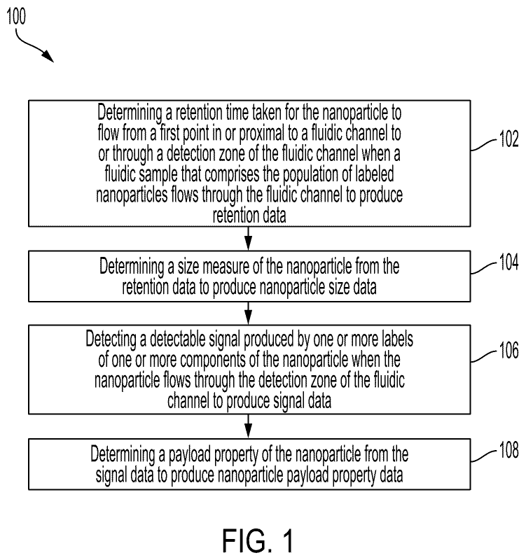

To illustrate some aspects of the methods disclosed herein, FIG. 1 is a flow chart that schematically shows exemplary method steps of characterizing a nanoparticle in a population of labeled nanoparticles according to some embodiments. As shown, method 100 includes determining a retention time taken for the nanoparticle to flow from a first point in or proximal to a fluidic channel to or through a detection zone of the fluidic channel when a fluidic sample that comprises the population of labeled nanoparticles flows through the fluidic channel to produce retention data (step 102). In some embodiments, the first point in or proximal to the fluidic channel comprises an inlet to the fluidic channel. Method 100 also includes determining a size measure of the nanoparticle from the retention data to produce nanoparticle size data (step 104). Method 100 also includes detecting a detectable signal produced by one or more labels of one or more components of the nanoparticle when the nanoparticle flows through the detection zone of the fluidic channel to produce signal data (step 106). In some embodiments, at least two, at least three, or more components of the nanoparticles in the population of nanoparticles comprise different labels from one another. In some embodiments, the different labels comprise different fluorescent labels. In addition, method 100 also includes determining a payload property of the nanoparticle from the signal data to produce nanoparticle payload property data (step 108). Typically, method 100 includes characterizing a plurality of the nanoparticles in the population of labeled nanoparticles in the fluidic sample. In some embodiments, for example, method 100 includes characterizing at least 2, at least 3, at least 4, at least 5, at least 10, at least 100, at least 10³, at least 10⁴, at least 10⁵, at least 10⁶, at least 10⁷, at least 10⁸, or more nanoparticles in a given population of labeled nanoparticles.

In some embodiments, at least a first component of the nanoparticles in the population of labeled nanoparticles comprises a payload molecule. In some embodiments, the payload molecule comprises a therapeutic or analytic agent, such as a nucleic acid vaccine, an siRNA, an antibody, and a small molecule, among many other payload types. In some embodiments, the payload molecule comprises a metabolite, a nucleic acid, and/or a polypeptide. In some embodiments, at least a second component of the nanoparticles in the population of labeled nanoparticles comprises a molecule selected from, for example, a polymer, a copolymer, a lipid, a fluorophore, a carbohydrate, an emulsion, a vesicle, a cell, polyethylene glycol (PEG), cholesterol, a liposome, a carbon nanotube, silica, gold, and the like. Other exemplary nanoparticle components are described further herein.

The methods disclosed herein are used to characterize various properties of nanoparticles in a population of labeled nanoparticles. In some embodiments, for example, the nanoparticle size measure comprises a diameter of the nanoparticle. In some embodiments, the at least one payload property of the nanoparticle comprises an amount of payload molecule, an encapsulation efficiency measure, and/or payload molecule capacity of the nanoparticle. In some embodiments, the retention data comprises a measure of hydrodynamic separation between the nanoparticle and at least one other nanoparticle in the population of labeled nanoparticles. In some embodiments, the signal data comprises a relative signal intensity and/or a coincidence measure of detectable signals produced by different labels of at least two components of the nanoparticles in the population of the labeled nanoparticles. In some embodiments, the method comprises distinguishing nanoparticles comprising a payload molecule from nanoparticles lacking a payload molecule in the population of the labeled nanoparticles using at least the signal data. In some embodiments, the method comprises determining a distribution of the at least one payload property of the nanoparticles in the population of labeled nanoparticles. In some embodiments, the method further comprises determining a concentration of nanoparticles in the population of labeled nanoparticles using at least the signal data. In some embodiments, the method comprises separating the population of labeled nanoparticles into at least two selected fractions before, during, or after characterizing the nanoparticle in the population of labeled nanoparticles. Typically, the methods disclosed herein comprise producing the nanoparticle size data and the nanoparticle payload property data substantially simultaneously.

In some embodiments, the method comprises illuminating the detection zone of the fluidic channel substantially uniformly across an entire cross section of the fluidic channel such that each of the nanoparticles in the population of labeled nanoparticles passes through illumination light upon passing through the detection zone. In some embodiments, the method comprises detecting each of the nanoparticles in the population of labeled nanoparticles based on corresponding responses to the illuminating to determine retention times taken for each of the labeled nanoparticles to flow from the first point in or proximal to the fluidic channel to or through the detection zone.

In some embodiments, the nanoparticles characterized using the methods disclosed herein are lipid nanoparticles (LNPs). LNPs can include numerous types of component molecules. In some embodiments, for example, LNPs include cationic lipids. Exemplary cationic lipids include, but are not limited to: N,N-dioleyl-N,N-dimethylammonium chloride (DODAC), 1,2-di-O-octadecenyl-3-trimethylammonium propane (DOTMA), N,N-distearyl-N,N-dimethylammonium (DDAB), 1,2-dioleoyl-3-trimethylammonium-propane (DOTAP, including chiral forms R-DOTAP and S-DOTAP), N-(1-(2,3-dioleyloxy)propyl)-N-2-(sperminecarboxamido)ethyl)-N,N-dimethylammonium (DOSPA), dioctadecylamidoglycyl carboxyspermine (DOGS), 1,2-dioleoyl-3-dimethylammonium propane (DODAP), N,N-dimethyl-(2,3-dioleyloxy)propylamine (DODMA), N-(1,2-dimyristyloxyprop-3-yl)-N,N-dimethyl-N-hydroxyethylammonium (DMRIE), 1,2-dilinoleyloxy-3-dimethylaminopropane (DLinDMA), 1,2-dilinolenyloxy-3-dimethylaminopropane (DLenDMA), 1,2-dilinoleoyl-3-dimethylaminopropane (DLinDAP), 1-linoleoyl-2-linoleyloxy-3-dimethylaminopropane (DLin-2-DMAP), 1,2-dilinoleylcarbamoyloxy-3-dimethylaminopropane (DLin-C-DAP), 1,2-dilinoleylthio-3-dimethylaminopropane (DLin-S-DMA), 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA), (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino)butanoate (DLin-MC3-DMA), 1,2-dipalmitoyl-sn-glycero-O-ethyl-3-phosphocholine (DPePC), distearyldimethylammonium chloride (DSDMA), 1,2-dilauroyl-sn-glycero-3-ethylphosphocholine (12:0 EPC, e.g., or a chloride salt thereof), 1,2-dipalmitoyl-sn-glycero-3-ethylphosphocholine (16:0 EPC, e.g., or a chloride salt thereof), 1,2-distearoyl-sn-glycero-3-ethylphosphocholine (18:0 EPC, e.g., or a chloride salt thereof), 1,2-dioleoyl-sn-glycero-3-ethylphosphocholine (18:1 EPC, e.g., or a chloride salt thereof), dipalmitoyl phosphatidylethanolamidospermine (DPPES), dipalmitoyl phosphatidyl ethanolamido L-lysine (DPPEL), 1-[2-dioleoyloxy)ethyl]-2-oleyl-3-(2-hydroxyethyl)imidazolinium chloride (DOTIM), and (1-methyl-4-(cis-9-dioleyl) methyl-pyridinium-chloride)) (SAINT). A number of commercial preparations of cationic lipids may be included in the nanoparticles. Such commercial preparations include, but are not limited to: LIPOFECTAMINE™ (a combination of DOSPA and DOPE) and LIPOFECTIN® (a combination of DOTMA and DOPE) from Invitrogen Corp.; and TRANSFECTAM® (a composition including DOGS) and TRANSFAST™ from Promega Corp.

In some embodiments, LNPs include anionic lipids. Exemplary anionic lipids include, but are not limited to: phosphatidylglycerols (PGs), cardiolipins (CLs), diacylphosphatidylserines (PSs), diacylphosphatidic acids (PAs), phosphatidylinositols (PIs), N-acylphosphatidylethanolamines (NAPEs), N-succinylphosphatidylethanolamines, N-glutarylphosphatidylethanolamines, lysylphosphatidylglycerols, and palmitoyloleoylphosphatidylglycerol (POPG), as well as different chiral forms (e.g., R or S forms), salt forms (e.g., a chloride, bromide, trifluoroacetate, or methanesulfonate salts), and mixtures thereof.

In some embodiments, LNPs include neutral lipids. Exemplary anionic lipids include, but are not limited to: ceramides, sphingomyelin (SM), diacylglycerols (DAGs), 1,2-distearoyl-sn-glycero-3-phosphocholine (DSPC, including chiral forms R-DSPC and S-DSPC), 1,2-dioleoyl-sn-glycero-3-phosphocholine (DOPC), 1,2-dipalmitoyl-sn-glycero-3-phosphocholine (DPPC), 1,2-dioleoyl-glycero-sn-3-phosphoethanolamine (DOPE), 1-palmitoyl-2-oleoyl-sn-glycero-3-phosphocholine (POPC), 1-palmitoyl-2-oleoyl-sn-glycero-3-phosphoethanolamine (POPE), 1,2-dipalmitoyl-sn-glycero-3-phosphoethanolamine (DPPE), 1,2-dimyristoyl-sn-glycero-3-phosphoethanolamine (DMPE), 1,2-distearoyl-sn-glycero-3-phosphoethanolamine (DSPE), 1,2-dielaidoyl-sn-glycero-3-phosphoethanolamine (DEPE), 1-stearoyl-2-oleoyl-sn-glycero-3-phosphoethanolamine (SOPE), 1,2-dilinoleoyl-sn-glycero-3-phosphocholine (DLPC), as well as different chiral forms (e.g., R or S forms), salt forms (e.g., a chloride, bromide, trifluoroacetate, or methanesulfonate salts), and mixtures thereof.

In some embodiments, nanoparticles include sterol derivatives, such as cholesterol, derivatives of cholestanol (e.g., cholestanone, cholestenone, or coprostanol); 3β-[-(N—(N′,N′-dimethylaminoethane)-carbamoyl]cholesterol (DC-cholesterol, e.g., a hydrochloride salt thereof); bis-guanidium-tren-cholesterol (BGTC); (2S,3S)-2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yloxy)carbonylamino)ethyl 2,3,4,4-tetrahydroxybutanoate (DPC-1); (2S,3S)-((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2,3,4,4-tetrahydroxybutanoate (DPC-2); bis((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2,3,4-trihydroxypentanedioate (DPC-3); and 6-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yloxy)oxidophosphoryloxy)-2,3,4,5-tetrahydroxyhexanoate (DPC-4).

In some embodiments, may be included in the nanoparticles, including, but not limited to: 1,2-dimyristoyl-sn-glycero-3-phosphoethanolamine-N-(carbonyl-methoxy-polyethylene glycol) (PEG-DMPE or DMPE-PEG) (e.g., 1,2-dimyristoyl-sn-glycero-3-phosphoethanolamine-N-(carbonyl-methoxy-polyethylene glycol-2000) (PEG-2000-DMPE or DMPE-PEG or DMPE-PEG2k)), 1,2-dipalmitoyl-sn-glycero-3-phosphoethanolamine-N-(carbonyl-methoxy-polyethylene glycol) (PEG-DPPE or DPPE-PEG), 1,2-distearoyl-sn-glycero-3-phosphoethanolamine-N-(carbonyl-methoxy-polyethylene glycol) (PEG-DSPE or DSPE-PEG), 1,2-dioleoyl-sn-glycero-3-phosphoethanolamine-N-(carbonyl-methoxy-polyethylene glycol) (PEG-DOPE or DOPE-PEG), 1,2-dimyristoyl-sn-glycerol-3-(methoxy-polyethylene glycol) (PEG-DMG or DMG-PEG) (e.g., 1,2-dimyristoyl-sn-glycerol-3-(methoxy-polyethylene glycol) (PEG-2000-DMG or DMG-PEG or DMG-PEG2k)), 1,2-dipalmitoyl-sn-glycerol-3-(methoxy-polyethylene glycol) (PEG-DPG or DPG-PEG), 1,2-distearoyl-sn-glycerol-3-(methoxy-polyethylene glycol) (PEG-DSG or DSG-PEG), 1,2-dioleoyl-sn-glycerol-3-(methoxy-polyethylene glycol) (PEG-DOG or DOG-PEG), 3-N-[(w-methoxypoly(ethylene glycol)2000)carbamoyl]-1,2-dimyristyloxy-propylamine (PEG-C-DMA), R-3-[(ω-methoxy poly(ethylene glycol)2000)carbamoyl)]-1,2-dimyristyloxlpropyl-3-amine (PEG-2000-C-DOMG), and PEG-ceramide conjugates

The nanoparticles can include any other component to aid in stabilizing the LNPs, reducing aggregation of LNPs, and/or delivering a therapeutic agent (e.g., an RNAi agent). Exemplary components include polyamide-lipid conjugates (ATTA-lipids) based on ω-amino (oligoethyleneglycol) alkanoic acid monomers; gangliosides (e.g., asialoganglioside GM1 or GM2; disialoganglioside GD1a, GD1a-NAcGal, GD1-b, GD2, or GD3; globoside, monosialoganglioside GM1, GM2, or GM3, tetrasialoganglioside GQ1b, and trisialoganglioside GT1a or GT1b); antioxidants (e.g., α-tocopherol or β-hydroxytoluidine); one or more surfactants (e.g., sorbitan monopalmitate or sorbitan monopalmitate, oily sucrose esters, polyoxyethylene sorbitane fatty acid esters, polyoxyethylene sorbitol fatty acid esters, polyoxyethylene fatty acid esters, polyoxyethylene alkyl ethers, polyoxyethylene sterol ethers, polyoxyethylene-polypropoxy alkyl ethers, block polymers and cetyl ether, as well as polyoxyethylene castor oil or hydrogenated castor oil derivatives and polyglycerine fatty acid esters, such as Pluronic®, Poloxamer®, Span®, Tween®, Polysorbate®, Tyloxapol®, Emulphor®, or Cremophor® (e.g., Cremophor® EL having a major component of glycerol-polyethyleneglycol ricinoleate with fatty acid esters of polyethylene glycol); one or more amphiphilic agents (e.g., vegetable oils, such as soybean oil, safflower oil, olive oil, sesame oil, borage oil, castor oil, and cottonseed oil; mineral oils and marine oils, hydrogenated and/or fractionated triglycerides from such sources; medium chain triglycerides (MCT-oils, e.g., Miglyol®), and various synthetic or semisynthetic mono-, di- or triglycerides, as well as acetylated monoglycerides, or alkyl esters of fatty acids, such isopropyl myristate, ethyl oleate or fatty acid alcohols, such as oleyl alcohol, cetyl alcohol; and one or more salts.

Typically, one or more components (e.g., lipids, PEG-lipid conjugates, payload molecules, and/or the like) of the nanoparticles characterized using the methods disclosed herein are labeled, e.g., to facilitate subsequent detection. In some embodiments, the components are labeled prior to nanoparticle formation. In certain embodiments, labels and component molecules are directly conjugated to one another (e.g., via single, double, triple or aromatic carbon-carbon bonds, or via carbon-nitrogen bonds, nitrogen-nitrogen bonds, carbon-oxygen bonds, carbon-sulfur bonds, phosphorous-oxygen bonds, phosphorous-nitrogen bonds, etc.). Optionally, a linker attaches the label to a given component molecule. A wide variety of linkers can be used or adapted for use in conjugating labels and nucleic acids or other component molecules.

Essentially any label is optionally utilized to label the component molecules described herein. In some embodiments, for example, the label comprises a fluorescent dye (e.g., a rhodamine dye (e.g., R6G, R110, TAMRA, ROX, etc.), a fluorescein dye (e.g., JOE, VIC, TET, HEX, FAM, etc.), a halofluorescein dye, a cyanine dye (e.g., CY3, CY3.5, CY5, CY5.5, etc.), a BODIPY® dye (e.g., FL, 530/550, TR, TMR, etc.), an ALEXA FLUOR® dye (e.g., 488, 532, 546, 568, 594, 555, 653, 647, 660, 680, etc.), a dichlororhodamine dye, an energy transfer dye (e.g., BIGDYE® v 1 dyes, BIGDYE® v 2 dyes, BIGDYE® v 3 dyes, etc.), Lucifer dyes (e.g., Lucifer yellow, etc.), CASCADE BLUE®, Oregon Green, and the like. Other labels optionally adapted for use in the methods disclosed herein include, e.g., biotin, weakly fluorescent labels (Yin et al. (2003) Appl Environ Microbiol. 69(7):3938, Babendure et al. (2003) Anal. Biochem. 317(1): 1, and Jankowiak et al. (2003) Chem Res Toxicol. 16(3):304), non-fluorescent labels, calorimetric labels, chemiluminescent labels (Wilson et al. (2003) Analyst. 128(5):480 and Roda et al. (2003) Luminescence 18(2):72), Raman labels, electrochemical labels, radioisotope labels, and bioluminescent labels (Kitayama et al. (2003) Photochem Photobiol. 77(3):333, Arakawa et al. (2003) Anal. Biochem. 314(2):206, and Maeda (2003) J. Pharm. Biomed. Anal. 30(6): 1725), among many others.

A large variety of linkers are available for linking labels to nucleic acids and other component molecules and will be apparent to one of skill in the art. A linker is generally of a structure that is sterically and electronically suitable for incorporation into a component molecule. Linkers optionally include, e.g., ether, thioether, carboxamide, sulfonamide, urea, urethane, hydrazine, or other moieties. To further illustrate, linkers generally include between about one and about 25 nonhydrogen atoms selected from, e.g., C, N, O, P, Si, S, etc., and comprise essentially any combination of, e.g., ether, thioether, amine, ester, carboxamide, sulfonamide, hydrazide bonds and aromatic or heteroaromatic bonds. In some embodiments, for example, a linker comprises a combination of single carbon-carbon bonds and carboxamide or thioether bonds. Although longer linear segments of linkers are optionally utilized, the longest linear segment typically contains between about three to about 15 nonhydrogen atoms, including one or more heteroatoms.

The present disclosure also provides various kit that includes a device comprising a fluidic channel having a detection zone (e.g., a microfluidic device, a capillary tube device, etc.), and instructions for using the device to: determine a retention time taken for a nanoparticle to flow from a first point in or proximal to the fluidic channel to or through the detection zone of the fluidic channel when a fluidic sample that comprises a population of labeled nanoparticles flows through the fluidic channel to produce retention data, determine a size measure of the nanoparticle from the retention data to produce nanoparticle size data, detect a detectable signal produced by one or more labels of one or more components of the nanoparticle when the nanoparticle flows through the detection zone of the fluidic channel to produce signal data, and determine at least one payload property of the nanoparticle from the signal data to produce nanoparticle payload property data. In some embodiments, the kit further comprises instructions for using the device to determine a concentration of nanoparticles in the population of labeled nanoparticles using at least the signal data. In some embodiments, the device comprises a microfluidic device.

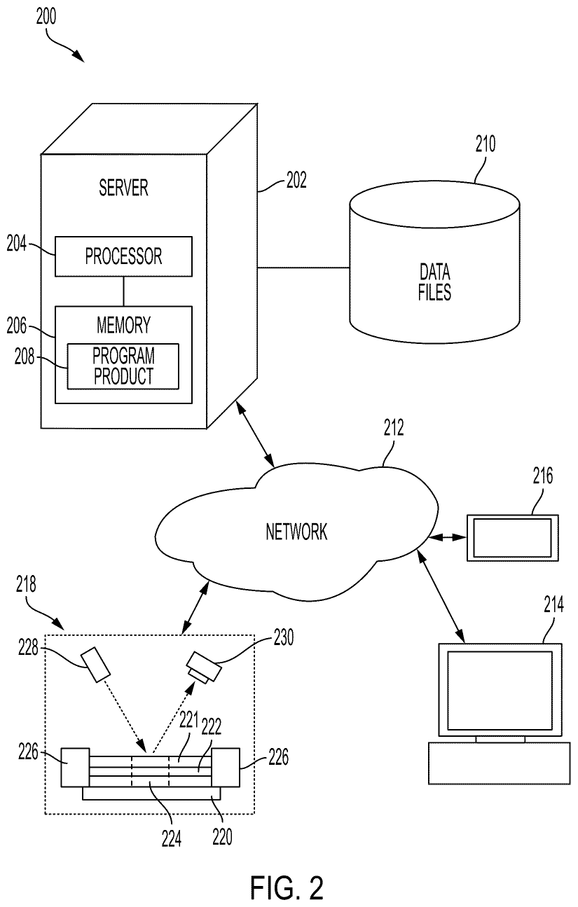

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving retention data, nanoparticle size data, signal data, nanoparticle payload property data, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for characterizing a nanoparticle in a population of labeled nanoparticles as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to retention data, nanoparticle size data, signal data, nanoparticle payload property data, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: determining a retention time taken for a nanoparticle to flow from a first point in or proximal to a fluidic channel to or through a detection zone of the fluidic channel when a fluidic sample that comprises a population of labeled nanoparticles flows through the fluidic channel to produce retention data, determining a size measure of the nanoparticle from the retention data to produce nanoparticle size data, detecting a detectable signal produced by one or more labels of one or more components of the nanoparticle when the nanoparticle flows through the detection zone of the fluidic channel to produce signal data, and determining at least one payload property of the nanoparticle from the signal data to produce nanoparticle payload property data.

In some embodiments, data for characterizing a nanoparticle in a population of labeled nanoparticles is obtained using sub-assembly 218. As shown, sub-assembly 218 includes device receiving area 220 configured to receive device 221 (e.g., a capillary tube, a microfluidic device, etc.) that includes fluidic channel 222 having detection zone 224. Sub-assembly 218 also includes fluid handling apparatus 226 (e.g., a syringe pump, a peristaltic pump, a pressure-driven pump, or the like) configured to effect a flow of a fluidic sample that comprises the population of labeled nanoparticles through detection zone 224 when fluid handling apparatus 226 is operably connected to device 221 and when the fluidic sample is disposed in fluidic channel 222. Sub-assembly 218 also includes light source 228 (e.g., one or more lasers, etc.) configured to introduce an incident light toward detection zone 224 when device 221 is received in device receiving area 220. As also shown, sub-assembly 218 also includes detector 230 (e.g., a CMOS camera, etc.) configured to detect one or more detectable signals (e.g., fluorescent signals) produced in detection zone 224 when fluid handling apparatus 226 effects the flow of the population of labeled nanoparticles flow through fluidic channel 222 and when device 221 is received in the device receiving area 220.

Some embodiments of the present disclosure are directed to hydrodynamic systems and methods for separating and determining sizes of particles in a sample. Some embodiments of the present disclosure can provide a method for single molecule analysis of size-separated LNP component molecules. By integrating cylindrical illumination confocal spectroscopy (CICS) according to embodiments of the present disclosure, size-specific single molecule analysis of LNP component molecules can be characterized. CICS has a sheet-like observation volume that enables substantially 100% detection efficiency of single molecules within the separation capillary in contrast to standard laser-induced fluorescence (LIF). In addition, direct single molecule counting can improve quantitative accuracy by eliminating reference curves and decoupling fluorescent intensity from abundance according to some embodiments of the present disclosure. Additional details related to CICS and other aspects that are optionally adapted for use with methods and other aspects of the present disclosure are also described in, for example, U.S. Patent Pub. No. US 2013/0167623, filed Mar. 13, 2013, and U.S. Pat. No. 8,248,609, which issued Aug. 21, 2012, which are both incorporated by reference in their entirety.

### Example: Payload Distribution and Capacity of mRNA Lipid Nanoparticles

### Results and Discussion

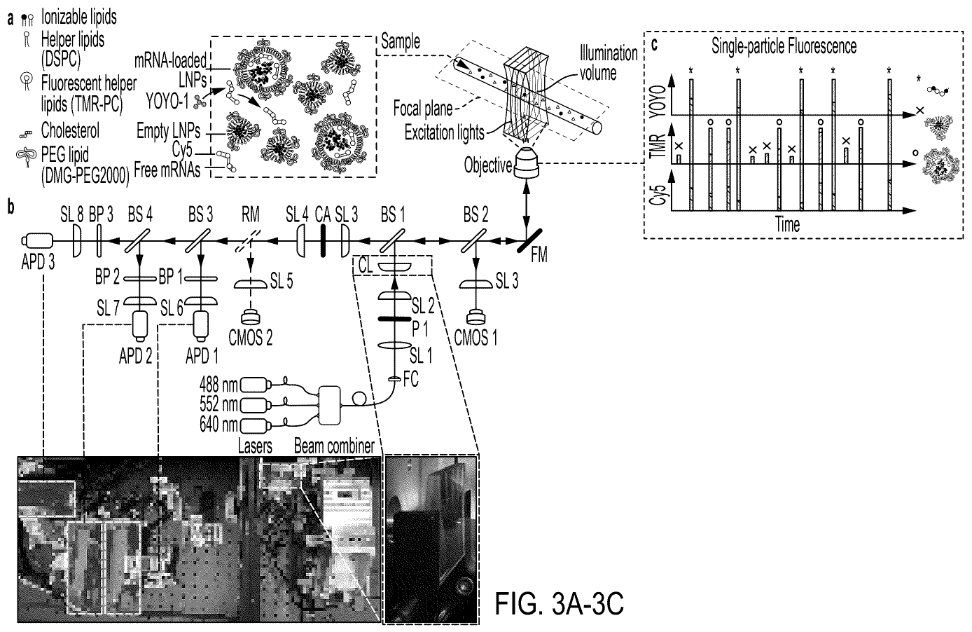

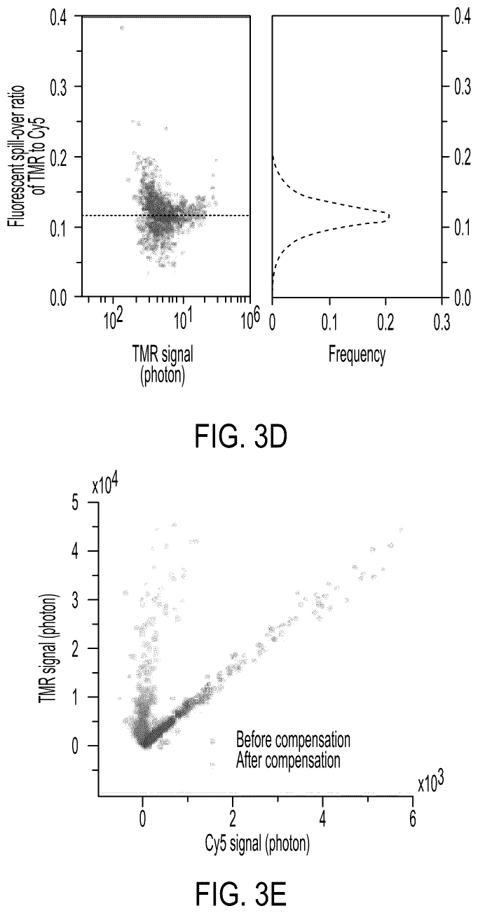

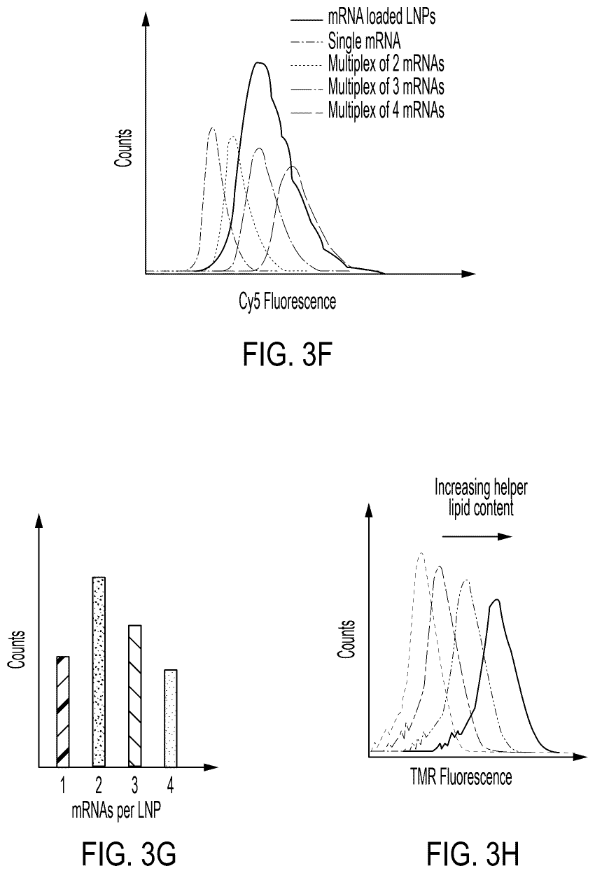

Methodology, multi-laser CICS instrumentation, fluorescent compensation, and deconvolution analysis. To track the mRNA in LNP samples (FIG. 3a), we used a commercially available Cy5-mRNA as the cargo that was 1,929 nucleotides in length. As it was synthesized through substituting 25% of uridine to Cy5-uridine during RNA polymerization, the molecules have a statistical distribution of Cy5 copies per mRNA, reflected as a base Cy5 signal profile for single mRNAs (FIG. 3f). LNPs loaded with multiple mRNAs generate higher levels of Cy5 signal, representing ensembles of different numbers of mRNA molecules, reflected as right-shifted histograms. A fluorescently labeled helper lipid, TMR-PC, was added at a molar ratio of 0.5% to tag all LNPs. Statistically, LNPs with a higher content of the helper lipid (DSPC) are expected to carry more TMR-PC thus a higher TMR signal (FIG. 3h). A nucleic acid-intercalating, lipid-impermeable dye YOYO-1 was added prior to CICS assessments to specifically stain un-encapsulated mRNAs.

The multi-color CICS platform was constructed as shown in FIG. 3b, (see Methods section for details). Concentration-optimized samples were introduced into a micron-sized capillary by a pressure-driven flow at a throughput of ~3000-5000 events/min that ensured one particle transits through the observation volume at a time. Three lasers with the wavelength matching the excitation spectra of fluorescent tags (488 nm, 552 nm, and 647 nm) were used for detection. The design of a cylindrical lens rendered a one-dimensional laser light sheet that covered the entire cross-section of the capillary, critical to a high fluorescence signal uniformity and mass detection efficiency. When passing the detection window, each LNP or free mRNA generated a unique fluorescent burst signal, which was captured with single-fluorophore sensitivity by CICS. The raw data were processed by a thresholding algorithm21 to identify and quantify these fluorescent bursts.

Common for multi-color fluorescence systems, fluorescent spillovers were observed in CICS, with TMR-to-Cy5 being the most significant. Compensation was therefore performed with single stained control samples. For example, we formulated TMR-PC-tagged empty LNPs with a large size and analyzed the distribution of the TMR-to-Cy5 spillover ratio across a wide range of signal intensity (FIG. 3d). A fixed ratio of 0.116 was then determined as the spillover ratio. The other channels were calibrated in the same way, and a compensation matrix (Table 1) was calculated as the inverse of the spill-over ratio matrix. After calibration, 93% of the spillover Cy5 signals from TMR fell below the lowest fluorescence given by individual mRNAs (i.e., 80 photons in burst size, FIG. 3e), and effectiveness was also confirmed in other channels. Upon compensation, different species of interest in an LNP formulation were determined by coincidence analysis of the fluorescence bursts (FIG. 3c, Table 2).

TABLE 1
Compensation matrix used in quantitative assessments

| | YOYO-1 | TMR | Cy5 |
|---|---|---|---|
| YOYO-1 | 1 | −0.071 | 0 |
| TMR | −0.033 | 1 | −0.116 |
| Cy5 | 0 | −0.007 | 1 |

TABLE 2
Differentiation strategy to distinguish different species in
LNP formulations based on fluorescence coincidence analysis

| | mRNA loaded LNPs | Empty LNPs | Free mRNA |
|---|---|---|---|
| YOYO-1 | − | − | + |
| TMR | + | + | − |
| Cy5 | + | − | + |

After identifying all mRNA-loaded LNPs, the mRNA payload in LNPs at the populational level can be estimated by comparing the mean Cy5 intensity of mRNA-loaded LNPs to that of the free mRNAs. However, the large variation in the fluorescence distribution prevents quantifying the payload for each LNP event. This variation is contributed by multiplicative factors that are inherent in the measurement, including mRNA payload capacity, Cy5 copy per mRNA, Possionian nature of photon emission and detection, and fluctuation of laser power and flow rate. As the factors except for mRNA payload capacity influence the measurement of LNPs and free mRNAs equally on CICS, it is then possible to quantify the mRNA payload capacity and its distribution by deconvolving the LNP Cy5 signal distribution against that of free mRNA (FIG. 3f). Detailed descriptions of the deconvolution analysis are in the Methods section. Briefly, the single mRNA fluorescence distribution DRNA,1 obtained by experiment was used to form the basis distributions DRNA, n|n=1, 2, . . . , N, which was generated by multiplying the fluorescence of DRNA,1 by n. DRNA, n represents the species of LNPs each containing exactly n mRNA molecules. DRNA, n|n=1, 2, . . . , N was used to construct an estimated LNP distribution DLNP* by assigning weights, wn, to each basis distributions DRNA, n.

$${D}_{LNP}^{*} = {\sum }_{n = 1}^{N} {w}_{n} \times {D}_{RNA , n}$$

The experimentally obtained LNP distribution, DLNP was deconvoluted into a linear combination of these weighted base distributions. The weights added up to be the estimated total number of mRNA-loaded LNPs, N*, which is the same as the experimental total number of mRNA-loaded LNPs N.

$$N = {N}^{*} = {\sum }_{n = 1}^{N} {w}_{n}$$

By tuning the weights to minimize the difference between DLNP* and DLNP, and an optimization factor given by χ2 (see Methods), the best fit DLNP* was determined. The weights, wn, in this best fit of DLNP* describes the distribution of the number of mRNAs encapsulated in LNPs (FIG. 3g).

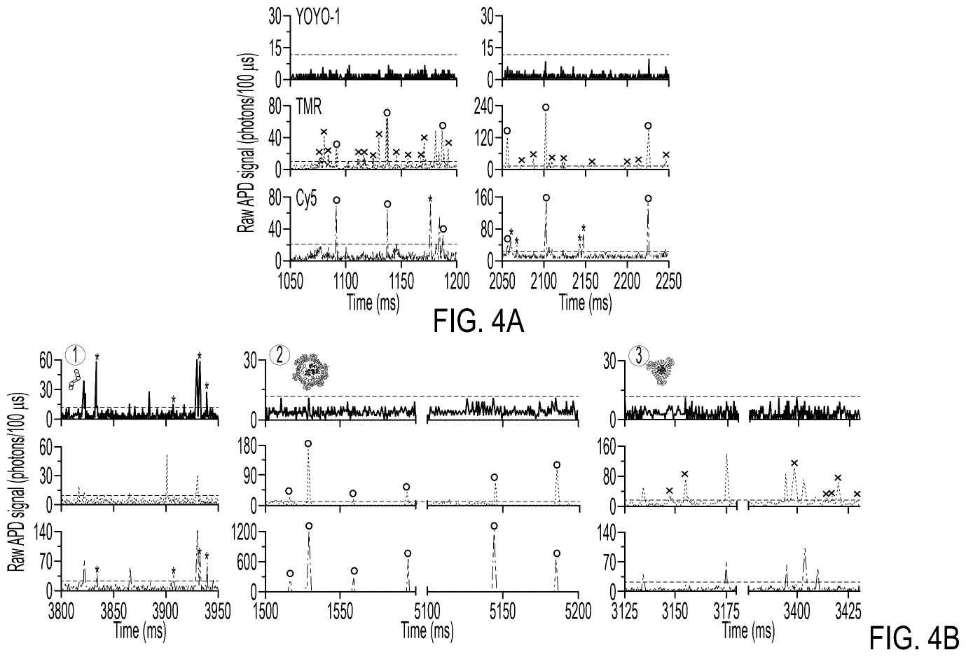

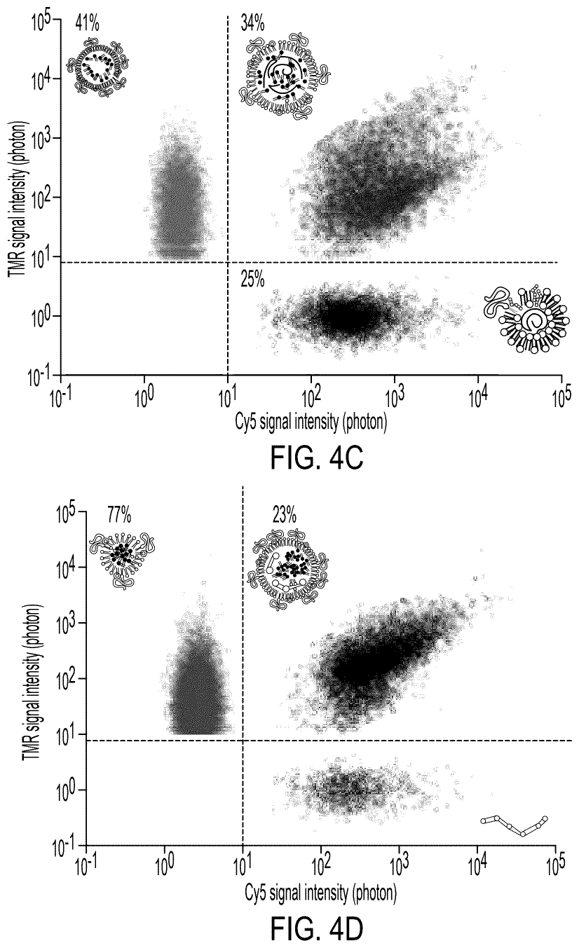

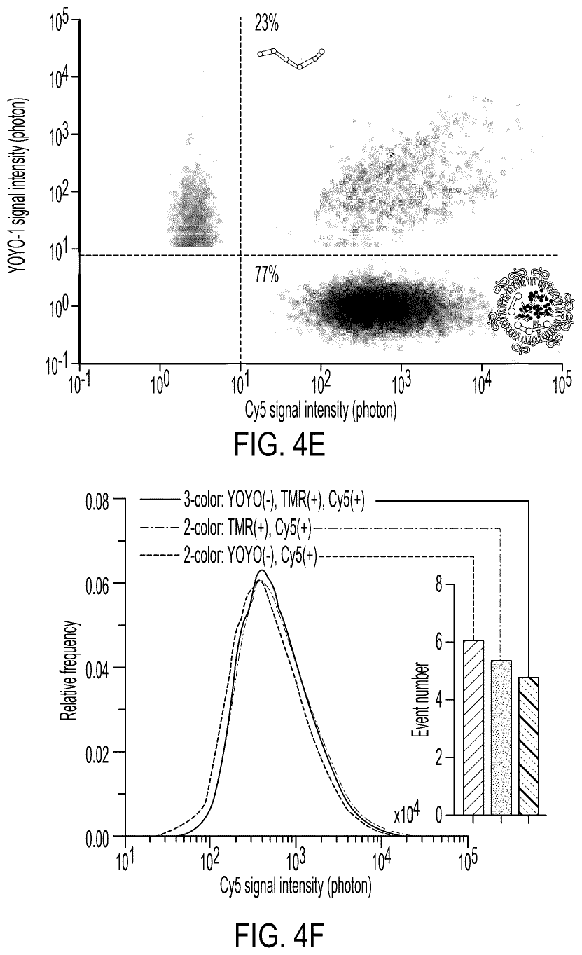

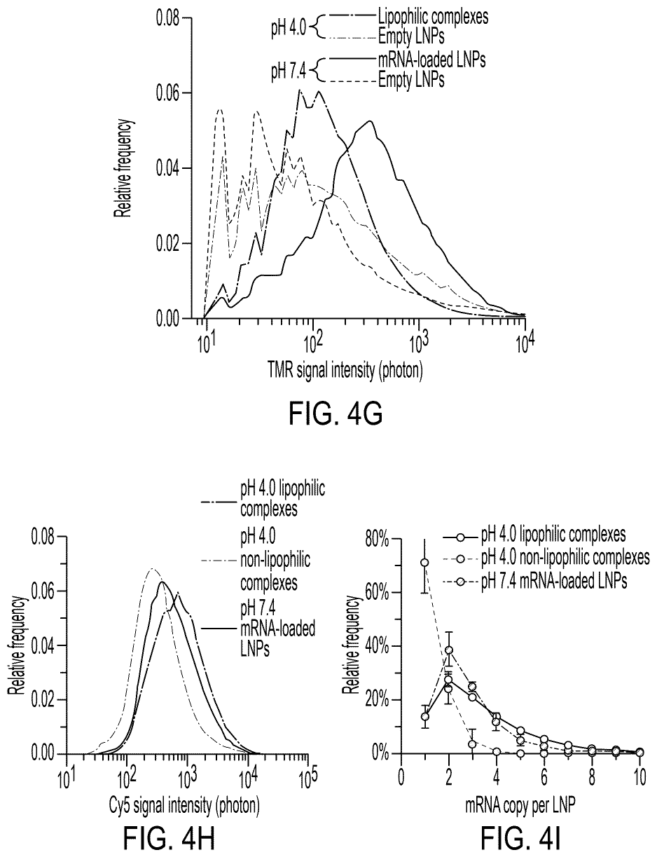

Characterization of a benchmark mRNA LNP formulation. Using the aforementioned methodology, we characterized a benchmark formulation prepared from a lipid mixture of DLin-MC3-DMA, 18:0 PC (DSPC), cholesterol, and DMG-PEG2000 at a molar ratio of 50:10:38.5:1.5. As the typical formulation process involves rapid mixing of lipids and mRNA solutions buffered at an acidic pH (in our study, 4.0) followed by dialysis against a buffer with pH 7.4, we sampled LNPs using 3-color CICS at both pHs to reveal the detailed payload characteristics of LNPs before and after dialysis (FIG. 4a, b). Considering a highly over-charged state of fully protonated ionizable lipids at pH 4.0, no YOYO-1 intercalation was observed (FIG. 4a). Plotting TMR vs. Cy5 signal intensities of all nanoparticle events allows clear identification of different populations in the LNP formulation. At pH 4.0 (FIG. 4c), three distinct populations were found: (1) TMR+ Cy5+ coincidences accounting for 34% of all events detected, which were presumably lipophilic mRNA complexes that contain a substantial amount of helper lipid. Note that the term “complexes” is used instead of “LNPs” to reflect the over-charged state; (2) TMR− Cy5+ signals accounting for 25% of all events detected, suggesting they might be non-lipophilic, highly charged complexes of mRNA and ionizable lipids that did not favor helper lipid insertion; (3) Cy5− TMR+ signals accounting for 41% of all events detected, which were empty LNPs, i.e., LNPs without an mRNA payload.

Following dialysis (pH 7.4, FIG. 4d), the fraction of empty LNPs was more substantial, accounting for 77% of all LNPs. Cy5+ TMR− events were unencapsulated mRNAs and accounted for only 4% of all events detected, which was expected for this formulation with a high encapsulation efficiency. The mRNA-loaded LNPs were identified by coincidence of TMR and Cy5 signals. TMR-Cy5 labeling scheme (FIG. 4d) effectively distinguished mRNA-loaded LNPs against free mRNAs; Nonetheless, the 3-color identification method with an additional YOYO-1 staining confirmation (FIG. 4e) was proved to be necessary to eliminate up to 10.4% of events found in the 2-color TMR/Cy5 labeling scheme that would have been falsely considered as mRNA-loaded LNPs (FIG. 4f, Table 2). Therefore, we used the 3-color identification method to achieve a higher degree of accuracy for calculating the payload distribution and capacity.

TMR fluorescence intensity profiles (as an indicator for relative helper lipid content, FIG. 4g) showed that lipophilic mRNA complexes at pH 4.0 or mRNA-loaded LNPs at pH 7.4 both contained a higher average helper lipid content than the empty LNPs. The comparison of the two conditions revealed a slight increase in helper lipid content from lipophilic mRNA complexes at pH 4.0 to mRNA-loaded LNPs at pH 7.4, corresponding to a decrease in helper lipid content in empty LNPs.

Using the deconvolution algorithm to analyze the fluorescence histograms of different LNP species (FIG. 4h), we depicted the mRNA payload distribution of the benchmark formulation (FIG. 4i). At pH 7.4, the number average mRNA payload was 2.80±0.41 among the mRNA-loaded LNPs, with around three quarters of them carrying 1 to 3 mRNAs per LNP. Based on all the data collected, a summary of the benchmark formulation is provided in Table 3.

TABLE 3
Composition features of the benchmark LNP formulation at
an mRNA concentration of 20 μg/mL and an N/P ratio of 6

| | Before dialysis at pH 4.0 | After dialysis at pH 7.4 |
|---|---|---|
| | (i.e., the initial LNPs) | (i.e., the initial LNPs) |
| Number-average payload | Lipophilic complexes: 3.43 ± 0.38 | 2.80 ± 0.41 |
| (mRNA copy per particle) | Non-lipophilic complexes: 1.34 ± 0.20 | |
| | All nanoparticles: 2.51 ± 0.24 | |
| Mode (most abundant) of | Lipophilic complexes: 2 | 2 |
| mRNA payload | Non-lipophilic complexes: 1 | |
| Populations | 34% ± 8% lipophilic complexes | 23% ± 8% mRNA-loaded LNPs |
| | 25% ± 4% non-lipophilic complexes | 77% ± 8% empty LNPs |
| | 41% ± 10% empty LNPs | |
| Particle number | Lipophilic complexes: | mRNA-loaded LNPs: |
| concentration* | 8.56 × 10¹⁵ ± 1.26 × 10¹⁵ mL⁻¹ | 1.29 × 10¹⁶ ± 2.22 × 10¹⁵ mL⁻¹ |
| | Non-lipophilic complexes: | empty LNPs: |
| | 6.47 × 10¹⁵ ± 9.74 × 10¹⁴ mL⁻¹ | 4.88 × 10¹⁶ ± 2.49 × 10¹⁶ mL⁻¹ |
| | Empty LNPs: | |
| | 1.11 × 10¹⁶ ± 5.04 × 10¹⁵ mL⁻¹ | |
| Encapsulation efficiency | N/A | 94.2% ± 3.6% by RiboGreen** |
| | | 85.6% ± 5.1% by CICS* |
| Average particle size*** | 106.3 ± 13.0 nm | 120.5 ± 6.0 nm |
| Zeta-potential*** | +45.1 ± 0.9 mV | −6.3 ± 1.3 mV |

Lipid composition: DLin-MC3-DMA:cholesterol:DSPC:DMG-PEG2000 = 50:38.5:10:1.5.
*The calculations for these parameters from CICS data are detailed in Supporting Information;
**The assay is described in Methods;
***The particle size is reported as z-average diameter assessed by dynamic light scattering (DLS), that counted all empty or mRNA-loaded LNPs. The zeta-potential was assessed by phase analysis light scattering (PALS).

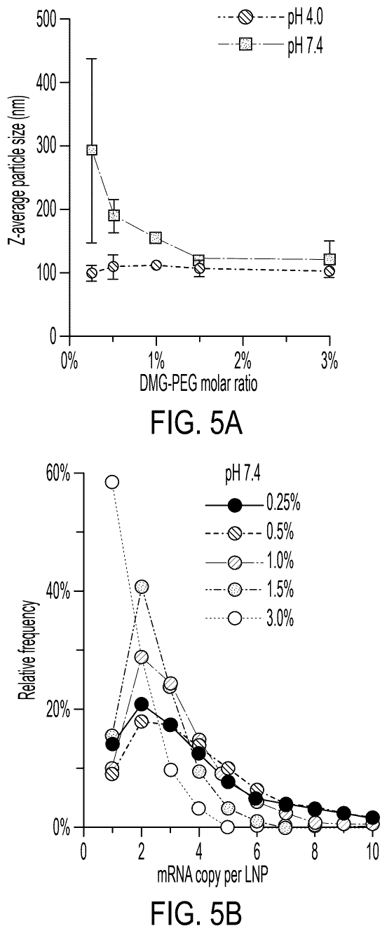

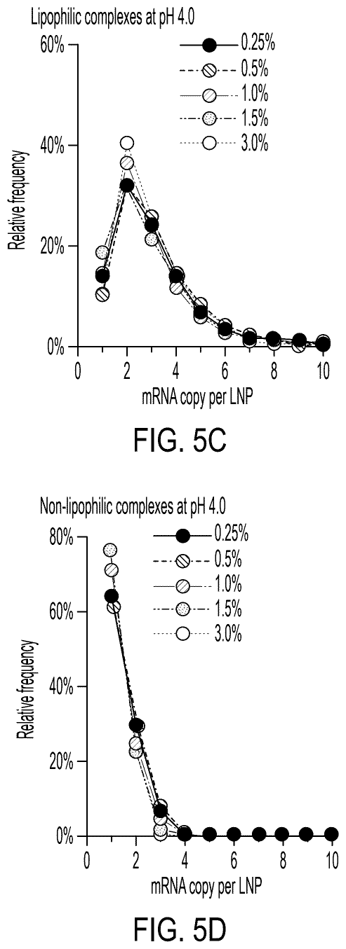

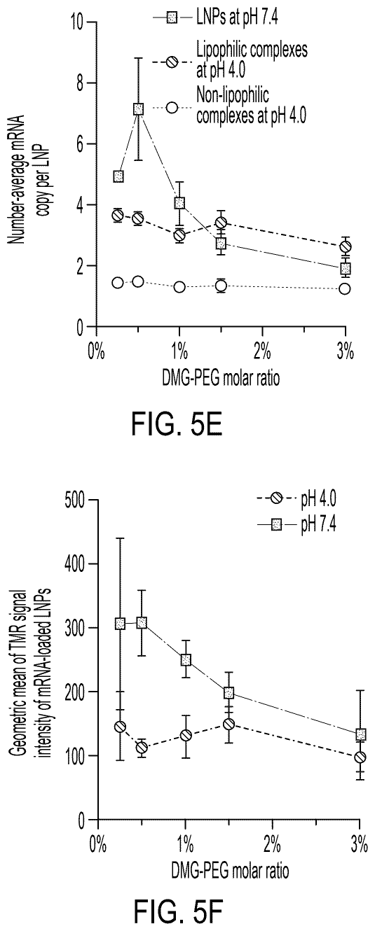

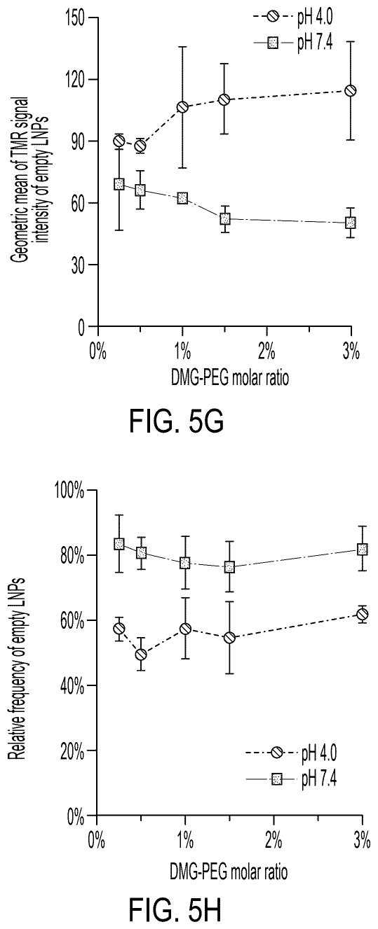

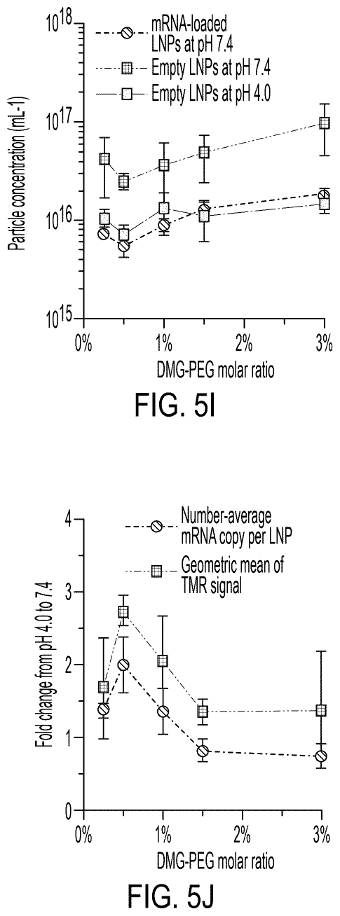

Effects of PEG lipid concentration on payload capacity of mRNA LNPs and composition drift during dialysis. Several reports demonstrated that the size of nucleic acid-loaded LNPs at pH 7.4 could be controlled by PEG lipid concentration. The measured size of LNPs and a theoretical “size limit” were correlated when a critical molecular area was assigned to the PEG lipid at LNP surfaces. When the mass content of other lipid components remains the same, a higher PEG % requires a higher surface-to-volume ratio, i.e., a smaller LNP size, to distribute the PEG lipids at a critical molecular surface density. In our experiments, by increasing the molar ratio of DMG-PEG2000 from 0.25% to 3%, the average LNP diameter at pH 7.4 decreased from 210 nm to 100 nm (FIG. 5a). The mRNA payload distributions of this LNP series analyzed by CICS clearly showed that the size difference directly correlates with the difference of payload capacity of mRNA LNPs (FIG. 5b, e). However, before dialysis at pH 4.0, the PEG concentration effect on LNP size was not observed (FIG. 5a), nor on mRNA payload distribution in either lipophilic or non-lipophilic mRNA complexes (FIG. 5c, d, e). These findings indicate that composition drifts occurred during dialysis due to deprotonation of ionizable lipids that transforms the LNPs from a state stabilized by surface PEG and an excess of residue positive surface charges at pH 4.0 to a state primarily stabilized by PEG at pH 7.4.

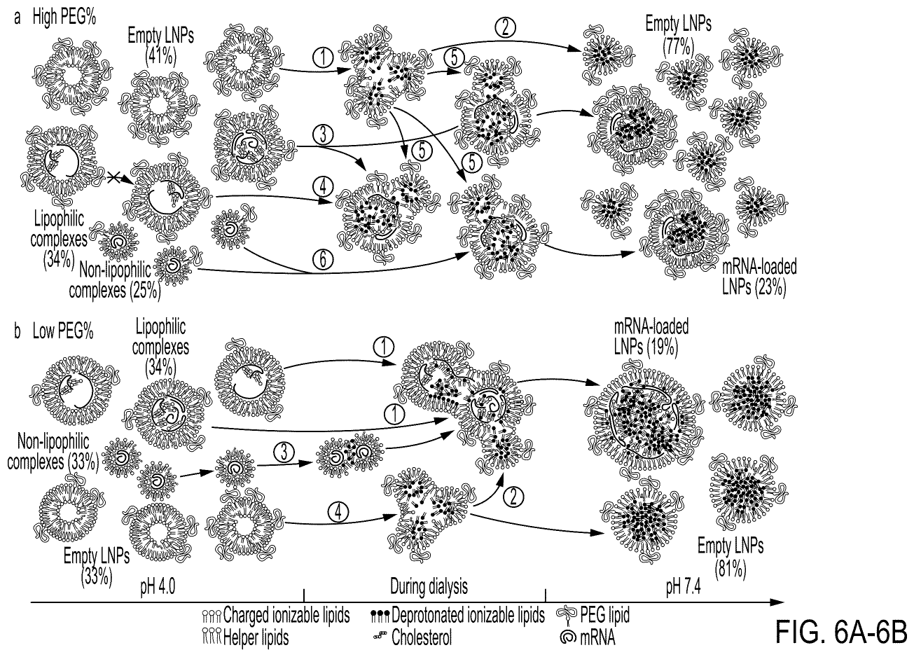

Our CICS data comparing the states at pH 4.0 and 7.4 suggest that during dialysis, empty LNPs split (FIG. 6a-1, 6b-4) as indicated by a drop in their average TMR signal intensity (FIG. 5g) and an increase in their concentration (FIG. 5h, i). Many empty particles remain mRNA-free until being stabilized at pH 7.4 (FIG. 6a-2). Since the helper lipid DSPC was reported to primarily reside on LNP surfaces, the driving force for splitting may be the transformation from a bilayer vesicle structure at pH 4.0 to a single layer surrounding a hydrophobic core of neutralized lipids at pH 7.4. This conversion requires an extra surface area to distribute helper lipid and can be realized by splitting. At pH 4.0, lipophilic complexes carried more mRNAs per LNP than non-lipophilic complexes (FIG. 5c, d, e). When PEG % is high (e.g., ≥1.5%), some lipophilic complexes with a high initial payload split to give lower payloads during dialysis (FIG. 5b, c, Fig. S6, FIG. 6a-3), whereas a large fraction of them with a medium or low initial payload maintained the same payload (FIG. 5b, c, FIG. 6a-4). When PEG % is very high (e.g., 3.0%), splitting of lipophilic complexes becomes dominant thus resulting in a lower average payload (FIG. 5e) and a higher LNP concentration (FIG. 5i). These complexes receive helper lipid content from merging with empty LNPs as indicated by an overall increase of TMR signal intensity after dialysis (FIG. 5f, j, FIG. 6a-5). This is presumably due to a lack of sufficient helper lipids and PEG lipids in the initial lipophilic complexes to stabilize these LNPs at pH 7.4. Because the payload distribution became relatively uniform at pH 7.4 (FIGS. 4d and 5b), non-lipophilic mRNA complexes mostly carrying a single or two mRNAs at pH 4.0 must have merged during dialysis (FIG. 5b, d, FIG. 6a-6). At the same time, they originally did not contain any helper lipid (TMR−, FIG. 4c), thus they must have received it from empty LNPs during dialysis (FIG. 6a-5). These analyses are consistent with FRET and cryo-EM observations in other reports, in which merging was considered as the major event during dialysis.

A low PEG content (e.g., s 1.0%) resulted in an increase in size limit at pH 7.4, enabling the lipophilic mRNA complexes to overcome the energy barrier to merge with each other (FIG. 6b-1), which significantly increased mRNA payload capacity (FIG. 5b, c, e). The LNPs received a significant amount of helper lipids from the empty LNPs during merging, as the fold increase of TMR signal was consistently found to be greater than that of mRNA payload after dialysis (FIG. 5j, FIG. 6b-2). Merging of non-lipophilic complexes (FIG. 6b-3) and splitting of empty LNPs (FIG. 6b-4) occurred in a similar manner as those with a higher PEG %.

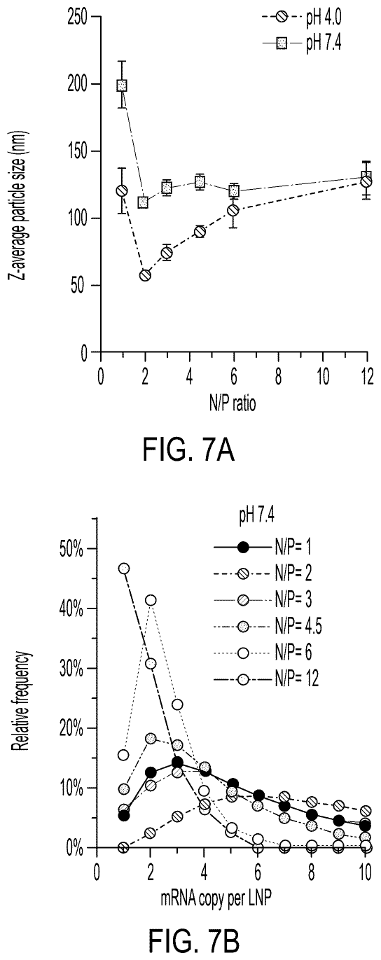

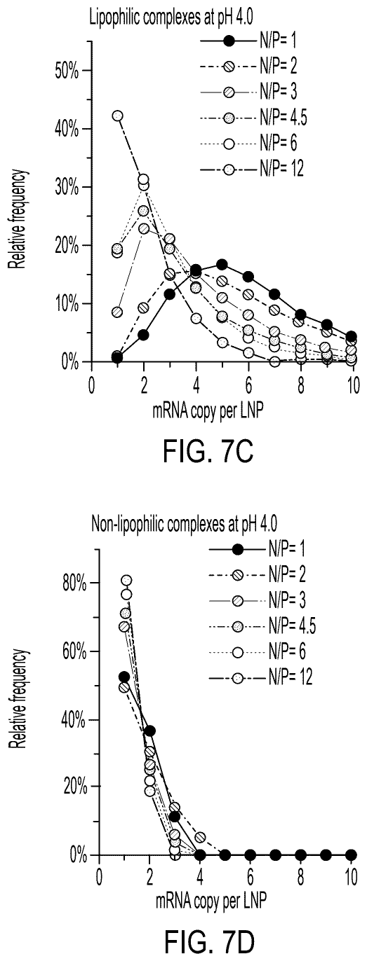

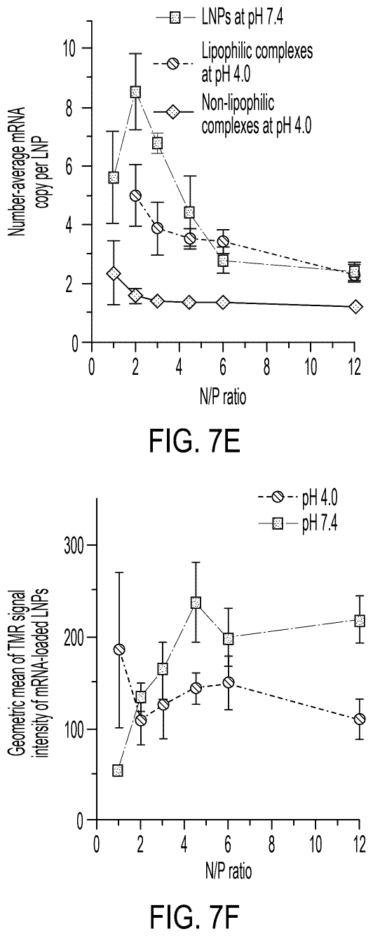

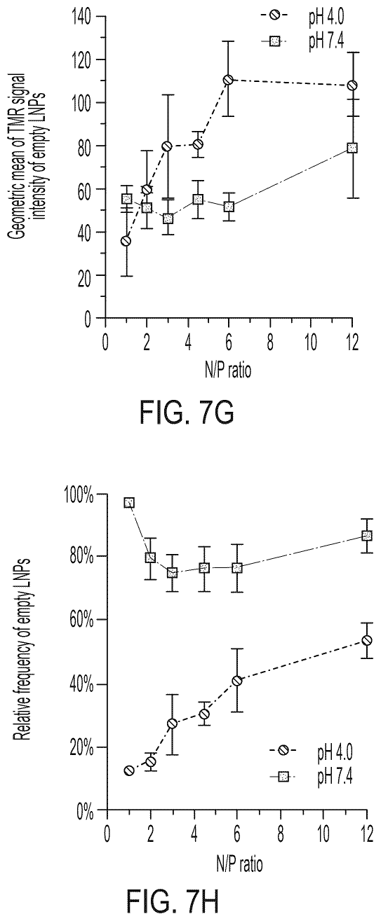

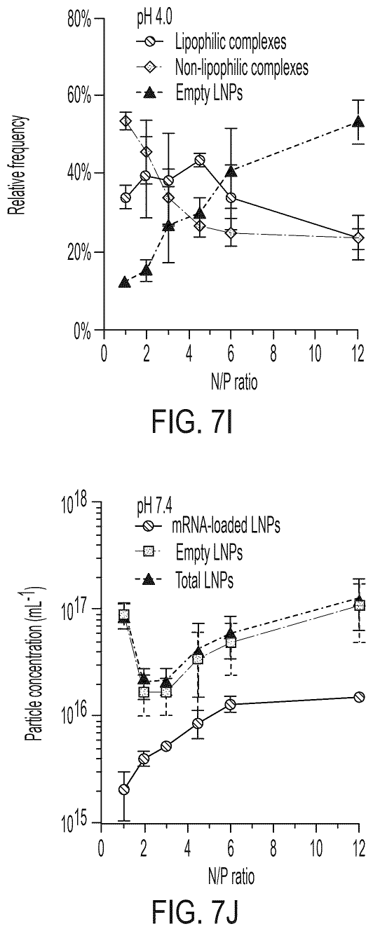

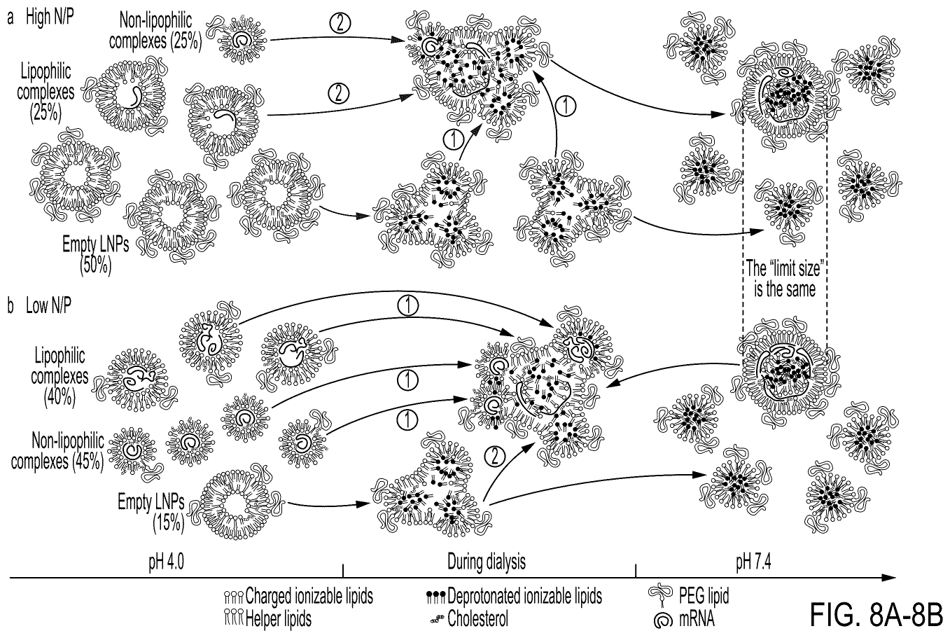

Effects of N/P ratio on payload capacity of mRNA LNPs and composition drift during dialysis. When N/P ratio (the molar ratio of amine groups on ionizable lipids to phosphate groups on mRNA) was tuned, the concentrations of all other lipid components were adjusted proportionally to that of ionizable lipid, while the mRNA concentration was kept consistent. This means that the PEG % to all lipids remained the same, yielding a consistent size of LNPs defined by the size limit correlated to the PEG surface density (FIG. 6a, FIG. 8). However, this same size permitted a higher mRNA payload per LNP as the N/P ratio decreased (FIG. 6b, e). The dynamic behaviors of different species during dialysis were again found to be essential for payload determination (FIG. 7e).

As the N/P ratio decreases, the relative lipid-to-mRNA mass ratio decreases, reducing the relative ratio of lipid mass incorporated into mRNA complexes to that incorporated into empty LNPs at pH 4.0. This was reflected as a strong positive correlation between the fraction of empty LNPs and the N/P ratio (FIG. 7h, i), but a negative correlation for the fraction of mRNA complexes (FIG. 7i). Therefore, when N/P ratio is high (FIG. 8a), fusion of empty LNPs with mRNA-loaded LNPs is the kinetically favorable events (FIG. 8a-1) until the mRNA-loaded LNPs are fully stabilized at the size limit defined by PEG %. Fusion of mRNA-carrying complexes appears to be minor (FIG. 8a-2), and consequently the final stabilized LNPs contain relatively fewer mRNA payloads. When N/P ratio is low, mRNA-loaded complexes are surrounded by more mRNA complexes than empty LNPs (FIG. 7i), and fusion of non-lipophilic complexes and between lipophilic complexes are kinetically favorable (FIG. 8b-1), whereas fusion of empty LNPs is less frequent (FIG. 8b-2). It is worth noting that mRNA complexes (lipophilic or non-lipophilic) also generally carried more copies of mRNA at pH 4.0 with a lower N/P ratio (FIG. 7c, d, e).

At pH 7.4, the mRNA-loaded LNPs contained a higher helper lipid content at a higher N/P ratio (FIG. 7f); while the empty LNPs shared a similar helper lipid content (FIG. 7g). A higher N/P ratio also generated a significantly higher concentration of LNPs (FIG. 7j).

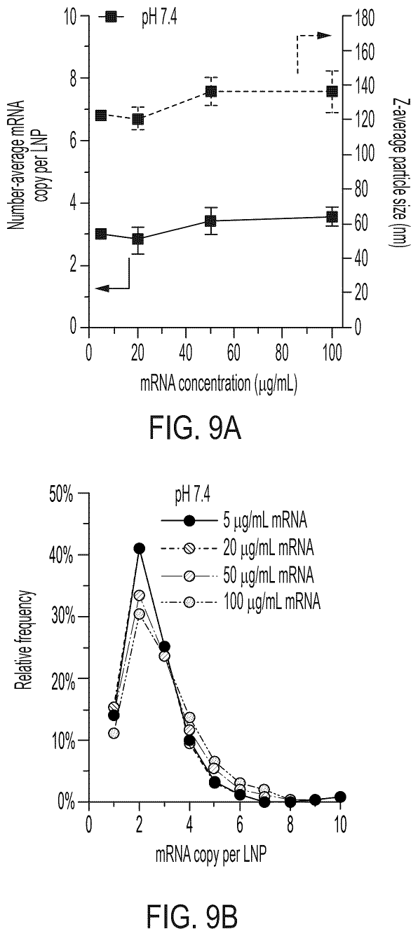

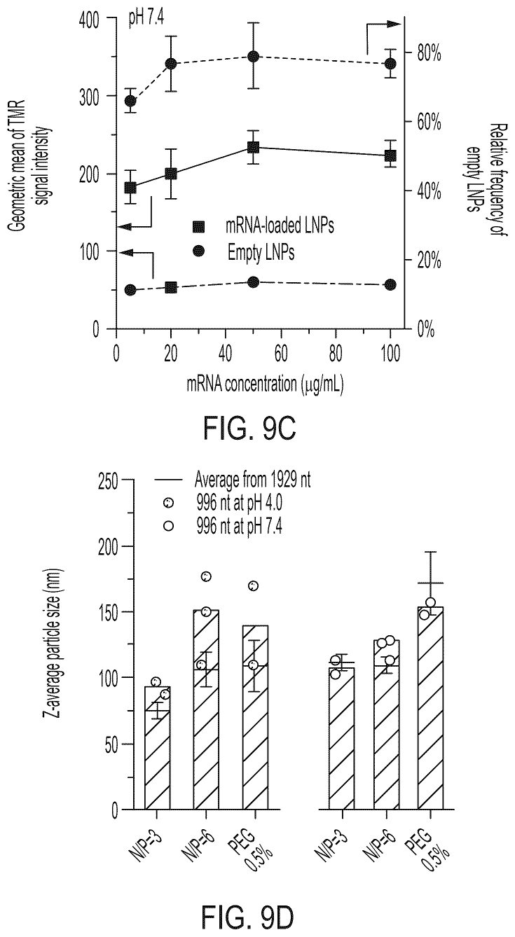

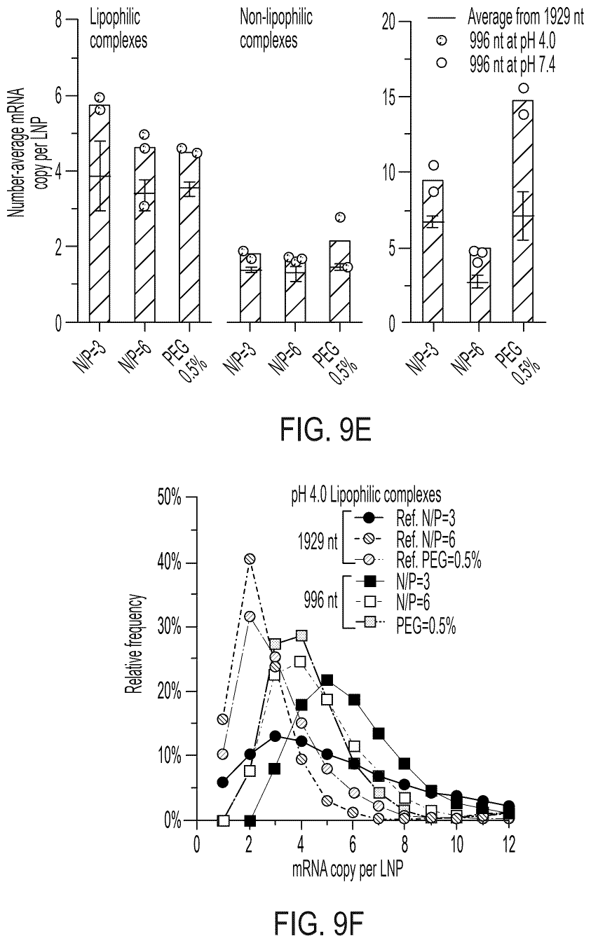

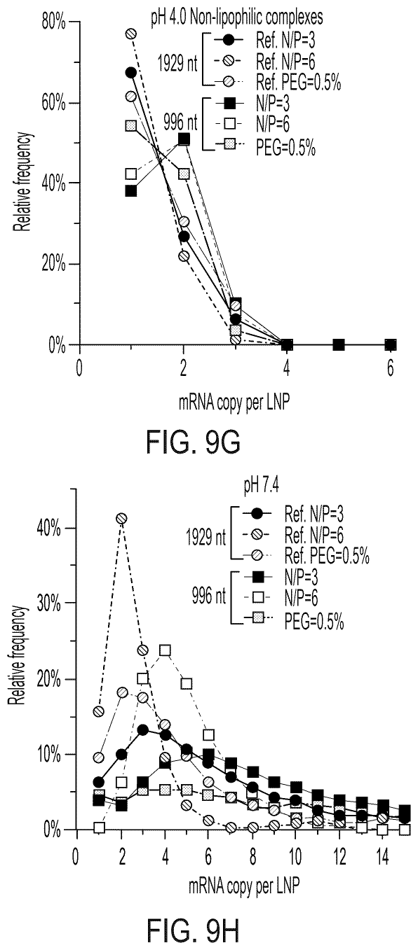

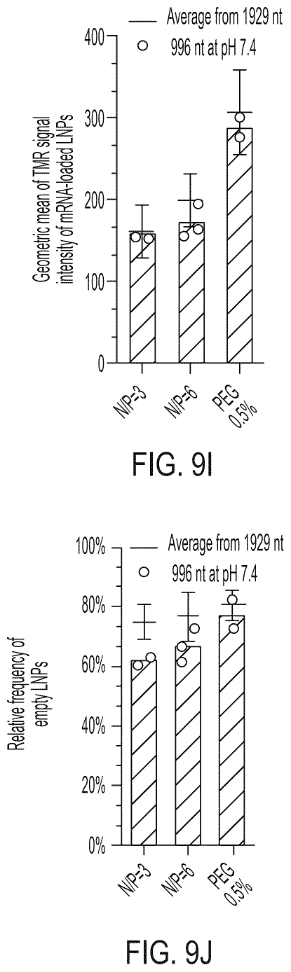

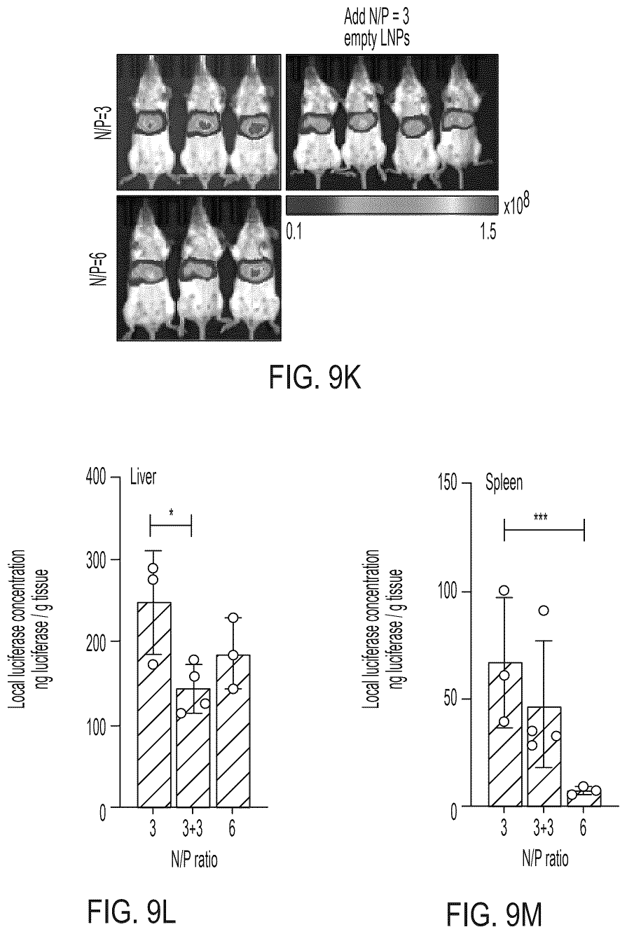

Effect of mRNA and lipid concentrations on payload capacity of mRNA LNPs. We next varied the mRNA concentration in the formulation from 5 to 100 μg/mL. The concentrations of all lipids were adjusted proportionally to maintain a constant relative lipid-to-mRNA mass ratio. Since the PEG % relative to all lipids remained constant, the same LNP size limit was observed at pH 7.4 (FIG. 9a). Therefore, the payload capacity and distribution profiles for LNP formulations in this series would remain the same; and this was verified by measured results as shown in FIG. 9a, b. The helper lipid content of mRNA-loaded LNPs and the fraction of empty LNPs appeared to be the lowest for the formulation with the lowest mRNA concentration of 5 μg/mL. Nonetheless, these metrics for all other formulations with 20-100 μg mRNA/mL were similar (FIG. 9c). At pH 4.0, these formulations yielded similar payload capacities of lipophilic and non-lipophilic complexes. This behavior was different from that of polyelectrolyte complexes of nucleic acids (e.g., pDNA/polyethyleneimine complexes), for which a higher overall nucleic acid concentration resulted in kinetic arrest of complexes with a higher pDNA payload per particle26. This difference may be explained by the high mobility of the cationic lipids as compared with polycations that ensures sufficient access to mRNA and charge neutralization, leading to effective formation of complexes with a lower degree of cross-complexation of multiple mRNA molecules.

Payload distribution and capacity of mRNA LNPs with different mRNA sizes. We next tested LNP formulations with a smaller mRNA (996 nt, half of the first mRNA) and examined 3 assembly conditions: N/P=3 and N/P=6 as those discussed in FIG. 7; and 0.5% PEG as that discussed in FIG. 5. Formulation of LNPs with the same mRNA mass concentration means a doubled number concentration for this 996-nt mRNA. At pH 7.4, change of mRNA size did not significantly affect the size limit of LNPs as a result of the same PEG % (FIG. 9d); however, the average payload significantly increased (FIG. 9e, h). The 2-fold reduction in mRNA size resulted in doubled payload in terms of the statistical mode (i.e., the most abundant) at pH 4.0 and 7.4 (FIG. 9f, g, h) for all LNP species. In the meantime, the helper lipid content of mRNA-loaded LNPs at pH 7.4 was similar between the two sets of LNPs with different mRNA sizes (FIG. 9i). These findings further support the conclusion that LNP assembly is most significantly influenced by the lipid concentrations or lipid-to-mRNA mass ratio, rather than mRNA concentrations. The copies of mRNA per LNP negatively correlated with the mRNA size, and it is approximated that the payload capacity of an LNP with a certain size limit can be reflected as a certain mass, instead of a certain number of mRNA.

Fraction of empty LNPs. Our analysis revealed that there was a significant fraction of empty LNPs in the final formulation at pH 7.4 for a wide range of conditions tested (FIGS. 5h, 7h). When the mRNA size was reduced from 1929 nt to 996 nt, the fraction of empty LNPs decreased at both N/P=3 and N/P=6 (FIG. 9j), which agrees with the prediction from a previous report that larger nucleic acid cargo tends to result in higher fraction of empty LNPs. With siRNA as cargo that was ~20 nt in size, a study found that there was no empty LNPs. We hypothesized that smaller nucleic acid molecules with higher diffusivity facilitate better mixing with the ionizable lipids, leading to more uniform complexation and effective reduction in co-packaging of multiple mRNAs in a single LNP, thus reducing the fraction of empty LNPs.

This finding has important biological implications for LNP-mediated gene delivery as the majority of LNPs dosed do not carry an mRNA payload. It leads to unnecessary exposure to a high amount of lipid components for the body. To explore the role of empty LNPs in intravenous (i.v.) mRNA delivery, we prepared two LNP formulations at N/P ratios of 3 and 6, both with around 75% empty LNPs, and compared them with an LNP formulation from mixing LNPs prepared at N/P=3 with additional empty LNPs to bring the total N/P ratio to 6 (contained 86% of empty LNPs; termed N/P=3+3). Following i.v. injection of the three LNPs carrying luciferase mRNA as a reporter gene in Balb/c mice, the luciferase expression in the liver was significantly reduced when the empty LNPs were added to the LNPs (FIG. 9k, I). The liver tropism of LNPs has been reported as a result of their interactions with apolipoprotein E (ApoE) in the blood, therefore the empty LNPs might have competed with mRNA-loaded LNPs for ApoE following injection and thus reduced delivery of mRNA to liver. When concerning gene expression in the spleen, only the mRNA-loaded LNPs from N/P=3 in both N/P=3 and N/P=3+3 groups, but not N/P=6, generated appreciable gene expression (FIG. 9k, m). The mRNA-loaded LNPs in N/P=6 held roughly half of mRNA payload comparing with those from N/P=3, with slightly higher helper lipid content but the same size (FIG. 7). These results indicated that the fraction of empty LNPs and payload capacity may influence the transgene expression profile following i.v. administration, although further investigations are needed to provide mechanistic insights.

### CONCLUSION

We developed a new single particle-analysis platform based on the CICS technique and reported for the first time the mRNA payload distribution and capacity, as well as the relative helper lipid content among different nanoparticle species in mRNA LNP formulations. This platform leverages the high sensitivity and versatility of the CICS technique as a powerful nanoparticle characterization tool. Using this method, we revealed that a benchmark mRNA LNP formulation contains mRNA-loaded LNPs mostly carrying 2 mRNAs in each particle with a number average of 2.8 mRNAs per LNP, and surprisingly, contains around 80% empty LNPs. We showed that the payload distribution and capacity were shaped from both the initial lipid phase separation and mRNA complexation at a low pH and compositional drifts during dialysis towards the physiological pH, in which the molar ratio of PEG lipids and lipid-to-mRNA mass ratio played a key role. The molar ratio of PEG lipids was found to dictate a size limit of the LNPs that positively correlated with the mRNA payloads, while the lipid-to-mRNA mass ratio controlled the fractions of the initial mRNA complexes vs. empty LNPs and kinetically influenced LNP fusion. We also revealed that the payload distribution and capacity were insensitive to the concentrations of mRNA and lipids, while the payload capacity of an LNP formulation likely correlated with a certain mass of nucleic acids thus that each LNP would contain a higher copy number of cargos with a smaller cargo size.

These findings provide impetus for further studies. For example, it will be intriguing to determine the factors controlling the complexation processes in forming lipophilic and non-lipophilic LNPs upon lipid/mRNA mixing and controlling the initial payload distribution. It will be helpful to understand how lipid compositions (e.g., different structures of the ionizable lipids, species of helper lipids and PEG lipids, and their relative ratios) influence the mRNA payload distribution and capacity. In addition, it will be critical to understand the effect of payload capacity and the fraction of empty LNPs on biodistribution, intracellular trafficking step (e.g., cellular uptake, endosomal escape, cargo release), and mRNA expression kinetics. These directions will raise the prospect of engineering new methods to control the payload distribution and capacity with desired lipid compositions and inspire further optimization of LNPs for the delivery of a wide range of nucleic acid therapeutics.

### Methods

Preparations of mRNA LNPs. The lipids used in this study were the same as a benchmark LNP formulation. The ionizable lipid DLin-MC3-DMA (MedKoo Biosciences, Cat #555308), the helper lipid 18:0 PC (DSPC, Avanti Polar Lipids, Cat #850365), cholesterol (Sigma-Aldrich, Cat #C8667), the PEG lipid DMG-PEG2000 (NOF America, Cat #GM020), and the fluorescent lipid TMR-PC (Avanti Polar Lipids, Cat #810180) were dissolved in pure ethanol with a molar ratio of 50:10:38.5:1.5:0.5. Cy5-mRNA (TriLink Biotechnologies, Cat #L-7702 with a length of 1929 nucleotides or Cat #L-7701 with a length of 996 nucleotides) was dissolved in 25 mM sodium acetate buffer at pH 4.0 to be accounted for an mRNA concentration of 5 to 100 μg/mL in the final LNP product. The molar ratio of the amine groups on the ionizable lipid to the phosphate groups on mRNA (i.e., the N/P ratio) was kept from 1 to 12. When mRNA concentration was altered, the N/P ratio was kept at 6; when N/P ratio was altered, the mRNA concentration was kept at 20 μg/mL while the concentrations of DSPC, cholesterol and DMG-PEG were altered proportionally to DLin-MC3-DMA. In the representative formulation (FIG. 4), the final mRNA concentration was 20 μg/mL with an N/P ratio of 6, correlating with 29 μg mRNA per μmol of total lipid components (including cholesterol). For formulating LNPs, a T-junction (IDEX Health and Science, Cat #P-890) was used. The lipid ethanol solution and the mRNA aqueous solution were injected into the T junction at a flow rate of 1 mL/min and 3 mL/min, respectively, controlled by two syringe pumps (New Era Pump Systems, Cat #NE-4000). The collected LNP suspension was dialyzed against 100-fold volume of 25 mM sodium acetate buffer at pH 4.0 (to remove ethanol) or phosphate buffered saline (PBS) at pH 7.4 (to remove ethanol and raise the pH to physiological pH) for 12 h under 4° C. by tubings with a molecular weight cut-off (MWCO) of 3,500 (Pur-A-Lyzer dialysis kit, Sigma-Aldrich, Cat #PURD35050). The LNPs were characterized immediately following dialysis.

Characterization of the size, zeta-potential and encapsulation efficiency of mRNA LNP formulations and optimization of YOYO-1 binding to free mRNA. Following dialysis, the size of the LNP formulations were assessed by dynamic light scattering (DLS, Malvern Zetasizer ZS90). The zeta-potential and the z-average diameter was reported for each formulation in this study. Quant-i™ RiboGreen assay (ThermoFisher Scientific, Cat #R11490) was used to characterize the encapsulation efficiency of the LNP formulations. Briefly, LNPs treated by 0.5% w/v Triton X-100 (Sigma Aldrich, Cat #T8787) to disrupt LNP structure and release mRNA and untreated LNPs were diluted to a concentration below 1 μg mRNA/mL, and then reacted with equal volume of RiboGreen assay solution at a 200-fold dilution. Standard curves were generated within 0.1 to 1.0 μg mRNA/mL using a series of free mRNA solutions with or without 0.5% w/v Triton X-100. The concentrations of free mRNA and total mRNA in the formulation were determined using bulk the fluorescent reading (excitation: 480 nm, emission: 520 nm) of the sample

against the corresponding standard curve. In CICS experiments, YOYO-1 iodide (ThermoFisher Scientific, Cat #Y3601) was used to stain unencapsulated mRNAs. To ensure highest detection sensitivity, the ionic strength of the PBS buffer at pH 7.4 and the molar ratio between YOYO-1 and mRNA were screened to yield a sensitivity over 95%, with 0.25-fold PBS and 1 nM YOYO-1 per 5 ng mRNA/mL being optimal, respectively. YOYO-1 was also used to characterize the encapsulation efficiency of LNP formulations. Different from RiboGreen, in which a fixed 200-fold diluted working assay solution was used for staining, the YOYO-1-to-mRNA ratio was kept consistent as 1 nM per 5 ng for both the sample and standard curves. The encapsulation efficiencies determined was found to be similar as those determined by RiboGreen.

Multi-color CICS instrumentation. The 3-color CICS was an expansion of the previous single-color version. A schematic of the optical setup is shown in FIG. 3a. The system contains three continuous wave lasers, with emission at 488 nm, 552 nm and 640 nm (OBIS LS 488-100FP, LS 552-80FP, LS 640-75FP, Coherent). The three lasers go through a laser beam combiner (OBIS Galaxy, Coherent) and output a single beam after an achromatic fiber collimator (f=4.0 mm, Thorlabs). The beam is expanded by a Keplerian beam expander which consists of two achromatic doublets (SL1, f1=19 mm and SL2, f2=75 mm, Thorlabs) and a 50-μm pinhole (Lenox Laser). The beam is further expanded in one dimension by a cylinderical lens (CL, f=150 mm, Thorlabs), and a dichroic mirror (FM, Thorlabs) is used to direct the excitation light into a 100× oil immersed objective (NA=1.3, Olympus) which also collects the emitted fluorescent signal from the sample. The sample is transported by a pressure driven flow in a fused silica microcapillary (Inner diameter=10 μm, Molex). The capillary is cut to be 50 cm in length and a transparent observation window is made by burning the polyimide coating on the exterior of the capillary at the length of 45 cm from the sample inlet. The capillary is mounted onto a glass slide and then placed onto a custom-made sample stage, which is further mounted onto a moterized XYZ stage (9063-XYZ-PPP-M, Newport). Two dichroic mirrors, DM1 and DM2 (LM01-552-25 and BLP01-635R-25, Semrock) are used to separate the signals induced by the three lasers. Then, the signals pass through a rectangular confocal aperture (CA, 292 μm×75 μm, National Aperture), which rejects the out-of-plane signal, and go through corresponding bandpass emission filters BP1, BP2, BP3 (FF02-520/28-25, FF03-575/25-25, and FF01-676/37-25, Semrock). The beams are then focused by doublets (SL5, SL6, SL7, f=30 mm, Thorlabs) onto the single-photon counting avalanche photodiodes (APD, SPCM-AQRH10, Excelitas). Two CMOS cameras (DCC3240C, Thorlabs) are used to accurately align the detection window to the microcapillary channel. A fraction of the light from the sample is directed by a pellicle beamsplitter (BS 2) and focused (SL 3) onto the first camera (CMOS 1) which guides the proper focus of the capillary. After the confocal aperture (CA), the second camera (CMOS 2) is used to accurately align the capillary position to be in the middle of the rectangular aperture, when the removable mirror (RM) is in place. During the experiment, a motorized flipper mount (8892-K-M, Newport) is used to switch off the mirror and direct the light to the APDs for data recording. A DAQ card (NI USB-6341, National Instruments) and a custom LabVIEW (Version 2020, National Instruments) are used for data acquisition at a rate of 250 kHz, with a bin size of 0.1 ms. The data analysis is performed on a laptop with custom MATLAB codes (Version 2021a, MathWorks).

Multi-color CICS experimental procedure. The LNP samples after dialysis in both sodium acetate buffer at pH 4.0 and phosphate buffered saline at pH 7.4 were further diluted in the corresponding buffer with 2% w/v PEG (20 kDa MW, Sigma Aldrich, Cat #81300). PEG was used as a dynamic coating additive to minimize adsorption in the capillary. After the encapsulation efficiency was determined, the free mRNA in the samples were stained with YOYO-1 iodide at a ratio of 1 nM YOYO-1 per 5 ng mRNA/mL. The mixture was incubated in PCR tubes in dark for at least 1 hour. The sample vial was placed in a pressure chamber and connected to the inlet end of the capillary. The sample was then injected into the capillary driven by a high pressure argon gas (AR HP6K, Airgas) at 42 psi, which gave a flow rate of 1 mm/s. For each LNP formulation, at least 50,000 signals were collected over a 20-min period for data analysis. A sample of free mRNA stained by YOYO-1 at a ratio of 1 nM YOYO-1 per 5 ng mRNA/mL in both pH 4.0 and pH 7.4 were analyzed to collect the basis Cy5 signal histogram for individual single mRNAs. After each sample run, the capillary was cleaned by flushing 0.1 M NaOH, deionized water, and 0.1 M HCl for three times, followed by corresponding buffer. Each capillary cleaning run went through at least 5 capillary lengths at 800 psi.

Single-nanoparticle analysis of mRNA LNPs using multi-color CICS. The single nanoparticle data analysis of CICS consists of three parts: single-particle fluorescence burst quantification, three-color coincidence detection for particle classification, and deconvolution analysis for mRNA payload characterization. The first part, single-particle fluorescence burst quantification, has been described in detail in our previous works. Briefly, the raw single fluorescence data were processed by a thresholding algorithm to identify the single-nanoparticle burst events. The information of each burst event including the retention time (ms), the start and end time of the burst (ms), burst height (photons/ms), burst width (ms), and burst size (photons) were recorded. These identified bursts in each color went through a coincidence detection algorithm which matched the coincidence events in two colors. The algorithm selected the burst events with their retention time difference between the two colors smaller than half of their maximum burst widths. The algorithm went through all the two-color combinations including Cy5-TMR, Cy5-YOYO-1, and TMR-YOYO-1. The burst size of the identified coincident events was adjusted by the compensation matrix before output for the analysis next step. For the sample in pH 4.0 buffer, the Cy5-TMR coincidence events were classified as lipophilic mRNA complexes; Cy5 without TMR events were classified as the non-lipophilic complexes; TMR without Cy5 events were classified as the empty LNPs. For the sample in pH 7.4 buffer, the Cy5-TMR coincident events without YOYO-1 were classified as mRNA-loaded LNPs; the Cy5-YOYO-1 coincident without TMR were classified as free mRNAs; the TMR without any coincident events were classified as the empty LNPs; the TMR-YOYO-1 coincident events were classified as the non-specific binding YOYO-1. The fluorescence distributions of the classified species were plotted and analyzed by their geometric mean. The distribution of the LNPs was further used for the payload capacity analysis by deconvolution algorithm.

Deconvolution of CICS signals to correlate with mRNA payload in LNPs. The fluorescence signal deconvolution algorithm was first proposed by Mutch et al. (Biophysical Journal 92, 2926-2943 (2007)) to process total internal reflection fluorescence (TIRF) microscopic images as a way to count protein number. In our previous work, we applied this analytical tool to quantify the DNA content distributions in PEI/DNA and PEI-g-PEG/DNA nanoparticles, and the same method was adopted for the mRNA payload analysis in this work. First, the fluorescence distributions of the mRNA-loaded LNPs species (DLNP) and the free mRNA (DRNA) were obtained by the CICS experiments and single particle analysis, and normalized by their total number of events. The distributions of the particle fluorescence were best described by a lognormal rather than the Gaussian distribution explained by the multiplicative processes, and thus quantified with logarithmic binning. By multiplying DRNA by a scaling factor (n), a set of basis distributions D_{RNA, n|n=1,2, . . . , N} were generated, which essentially represents a set of monodispersed particles each containing exactly n mRNA molecules. To maximize the computation accuracy, the upper limit (Nₘₐₓ) of the scaling factor was chosen to be six times the average number of mRNA per particle, which was estimated by the ratio of the geometric mean of fluorescence distribution of the mRNA-loaded LNPs to that of the free mRNAs.

$${{\sum }_{i = 1}^{{I}_{B}} {D}_{RNA , n} ( i ) = 1 \vert}_{n = 1 , 2 , … , N}$$

$${N}_{max} = 6 * \frac{{μ}_{LNP}}{{μ}_{mRNA}}$$

D_{RNA, n}(i) represents the proportion of each distribution in ith bin, for all n. I_{B} is the number of bins for each distribution. D_{LNP} was estimated by a fitted distribution, D_{LNP}*, which was constructed by assigning weights, wₙ, to each basis distributions D_{RNA, n}. n_{LNP} is the total number of events in the LNP distribution.

$${D}_{LNP}^{*} = {\sum }_{n = 1}^{N} {w}_{n} \times {D}_{RNA , n}$$

$$0 \leq {w}_{n} \leq {n}_{LNP}$$

A fitted estimate mRNA LNP distribution, D_{LNP}* is constructed by assigning weights, wₙ, to each basis distributions D_{RNA, n}, whereas D_{LNP} is the mRNA LNP distribution obtained experimentally which is deconvoluted into a linear combination of the weighted basis distributions.

For each single bin, we have

$${y}_{i}^{*} = {D}_{LNP}^{*} ( i ) = {\sum }_{n = 1}^{N} {w}_{n} \times {D}_{RNA , n} ( i )$$

Where yᵢ* is the estimated number of the mRNA-loaded LNPs in the ith bin and the estimated total number of mRNA-loaded LNPs, N*, is given by

$${N}^{*} = {\sum }_{n = 1}^{N} {w}_{n}$$

The difference between the constructed mRNA LNP distribution, D_{LNP}*, and the experimental D_{LNP} is χ2.

$${𝒳}^{2} = ( {\sum }_{i = 1}^{{I}_{B}} \frac{{( {y}_{i} - {y}_{i}^{*} )}^{2}}{{y}_{i}} ) + {α ( {N}_{LNP} - {N}^{*} )}^{2}$$

where α is a penalty factor imposed in the optimization. α was chosen to be 0.1 to ensure N. is less than 1% off from N_{LNP}. The goal of the deconvolution analysis is to minimize χ2 by finding the best weight assigned to the basis distributions, wₙ to describe the mRNA payload in the LNP sample. This optimization was performed by a simulated annealing algorithm in Matlab. All the source codes of the aforementioned data analysis can be provided upon request.

Animal model. The intravenous (i.v.) LNP delivery experiments were approved by the Johns Hopkins Animal Care and Use Committee (ACUC, protocol #MO20E63). The LNP formulations carrying luciferase mRNA (TriLink Biotechnologies, Cat #L-7202) were formulated as described above and dialyzed into PBS buffer, and subsequently injected through the tail lateral vein at a dose of 0.5 mg mRNA/kg. At 12-hour post-injection, 100 μL of D-luciferin solution (25 mg/mL in PBS, Gold Biotechnology, Cat #LUCK) was intraperitoneally injected into each mouse. The mice were imaged by an IVIS live-animal imaging system (Perkin Elmer) 5 minutes after injection. The liver and spleen were then harvested, weighted and digested by reporter lysis buffer (Promega, Cat #E4030) assisted by an ultrasonic processor (Qsonica, Cat #Q55A). The digested solution was subjected to a freeze-thaw cycle to fully release luciferase. The luciferase concentration within each organ sample was characterized by a standard luciferase assay (Promega, Cat #1500).

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of characterizing a nanoparticle in a population of labeled nanoparticles, the method comprising:
determining a retention time taken for the nanoparticle to flow from a first point in or proximal to a fluidic channel to or through a detection zone of the fluidic channel when a fluidic sample that comprises the population of labeled nanoparticles flows through the fluidic channel to produce retention data;
determining a size measure of the nanoparticle from the retention data to produce nanoparticle size data;
detecting a detectable signal produced by one or more labels of one or more components of the nanoparticle when the nanoparticle flows through the detection zone of the fluidic channel to produce signal data; and,
determining at least one payload property of the nanoparticle from the signal data to produce nanoparticle payload property data, thereby characterizing the nanoparticle in the population of labeled nanoparticles.

2. The method of claim 1, wherein the first point in or proximal to the fluidic channel comprises an inlet to the fluidic channel.

3. The method of claim 1, wherein the population of labeled nanoparticles comprises lipid nanoparticles.

4. The method of claim 1, wherein at least two, at least three, or more components of the nanoparticles in the population of nanoparticles comprise different labels from one another.

5.-8. (canceled)

9. The method of claim 1, wherein at least a first component of the nanoparticles in the population of labeled nanoparticles comprises a payload molecule.

10. (canceled)

11. (canceled)

12. The method of claim 1, wherein at least a second component of the nanoparticles in the population of labeled nanoparticles comprises a molecule selected from the group consisting of: a polymer, a copolymer, a lipid, a fluorophore, a carbohydrate, an emulsion, a vesicle, a cell, polyethylene glycol (PEG), cholesterol, a liposome, a carbon nanotube, silica, and gold.

13-18. (canceled)

19. The method of claim 1, comprising distinguishing nanoparticles comprising a payload molecule from nanoparticles lacking a payload molecule in the population of the labeled nanoparticles using at least the signal data.

20. The method of claim 1, comprising determining a distribution of the at least one payload property of the nanoparticles in the population of labeled nanoparticles.

21. The method of claim 1, further comprising determining a concentration of nanoparticles in the population of labeled nanoparticles using at least the signal data.

22. (canceled)

23. (canceled)

24. A kit, comprising:
a device comprising a fluidic channel having a detection zone; and,
instructions for using the device to:
determine a retention time taken for a nanoparticle to flow from a first point in or proximal to the fluidic channel to or through the detection zone of the fluidic channel when a fluidic sample that comprises a population of labeled nanoparticles flows through the fluidic channel to produce retention data;
determine a size measure of the nanoparticle from the retention data to produce nanoparticle size data;
detect a detectable signal produced by one or more labels of one or more components of the nanoparticle when the nanoparticle flows through the detection zone of the fluidic channel to produce signal data; and
determine at least one payload property of the nanoparticle from the signal data to produce nanoparticle payload property data.

25. The kit of claim 24, further comprising instructions for using the device to determine a concentration of nanoparticles in the population of labeled nanoparticles using at least the signal data.

26. (canceled)

27. (canceled)

28. A system for characterizing a nanoparticle in a population of labeled nanoparticles, comprising:
a device receiving area configured to receive a device comprising a fluidic channel having a detection zone;
a fluid handling apparatus configured to effect a flow of a fluidic sample that comprises the population of labeled nanoparticles through the detection zone when the fluid handling apparatus is operably connected to the device and when the fluidic sample is disposed in the fluidic channel;
a light source configured to introduce an incident light toward the detection zone when the device is received in the device receiving area;
a detector configured to detect one or more detectable signals produced in the detection zone when the fluid handling apparatus effects the flow of the population of labeled nanoparticles flow through the fluidic channel and when the device is received in the device receiving area;
a controller that comprises, or is capable of accessing, computer readable media comprising non-transitory computer-executable instructions which, when executed by at least one electronic processor, perform at least:
flowing the fluidic sample through the fluidic channel using the fluid handling apparatus such that the population of labeled nanoparticles flow through the detection zone of the fluidic channel;
introducing the incident light from the light source toward the detection zone when the device is received in the device receiving area;
determining a retention time taken for the nanoparticle to flow from a first point in or proximal to a fluidic channel to or through the detection zone of the fluidic channel when the population of labeled nanoparticles flows through the fluidic channel to produce retention data;
determining a size measure of the nanoparticle from the retention data to produce nanoparticle size data;
detecting a detectable signal produced by one or more labels of one or more components of the nanoparticle when the nanoparticle flows through the detection zone of the fluidic channel to produce signal data; and
determining at least one payload property of the nanoparticle from the signal data to produce nanoparticle payload property data to thereby characterize the nanoparticle in the population of labeled nanoparticles.

29.-32. (canceled)

33. The system of claim 28, wherein the light source comprises a cylindrical illumination apparatus.

34. The system of claim 28, wherein at least two, at least three, or more components of the nanoparticles in the population of nanoparticles comprise different labels from one another.

35. (canceled)

36. (canceled)

37. The system of claim 28, wherein the non-transitory computer-executable instructions which, when executed by the electronic processor, further perform at least: determining a concentration of nanoparticles in the population of labeled nanoparticles using at least the signal data.

38. (canceled)

39. The system of claim 28, wherein the non-transitory computer-executable instructions which, when executed by the electronic processor, further perform at least: producing the nanoparticle size data and the nanoparticle payload property data substantially simultaneously.

40.-43. (canceled)

44. The system of claim 28, wherein the non-transitory computer-executable instructions which, when executed by the electronic processor, further perform at least: illuminating the detection zone of the fluidic channel substantially uniformly across an entire cross section of the fluidic channel such that each of the nanoparticles in the population of labeled nanoparticles passes through illumination light upon passing through the detection zone.

45. The system of claim 28, wherein the non-transitory computer-executable instructions which, when executed by the electronic processor, further perform at least: detecting each of the nanoparticles in the population of labeled nanoparticles based on corresponding responses to the illuminating to determine retention times taken for each of the labeled nanoparticles to flow from the first point in or proximal to the fluidic channel to or through the detection zone.

46.-48. (canceled)

49. The system of claim 28, wherein the non-transitory computer-executable instructions which, when executed by the electronic processor, further perform at least: distinguishing nanoparticles comprising a payload molecule from nanoparticles lacking a payload molecule in the population of the labeled nanoparticles using at least the signal data.

50. (canceled)

51. (canceled)

52. The system of claim 28, wherein the non-transitory computer-executable instructions which, when executed by the electronic processor, further perform at least: determining an encapsulation efficiency measure of nanoparticles in the population of labeled nanoparticles.
